SEQUENCE LISTING (1 sequence)

SEQ ID NO: 1 - protein, 5 aa, Artificial sequence
  chemically synthesized FLNA sequence that corresponds to amino acid residue positions 2561-2565 of the FLNA protein
VAKGL

(19) United States
(12) Patent Application Publication
(10) Pub. No.: US 2010/0280057 A1
(43) Pub. Date: Nov. 4, 2010

(54) NOVEL ANALGESIC THAT BINDS FILAMIN A

(76) Inventors: Lindsay Burns Barbier, Palo Alto, CA (US); Hoau-Yan Wang, Philadelphia, PA (US); Nan-Horng Lin, Vernon Hills, IL (US); Andrei Blasko, San Bruno, CA (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 12/435,304

(22) Filed: May 4, 2009

(51) Int. Cl. A61K 31/438; C07D 413/02; A61P 29/00

(52) U.S. Cl. 514/278; 546/19

(57) ABSTRACT

A compound, composition and method are disclosed that can provide analgesia. A contemplated compound has a structure that corresponds to Formula I, wherein R¹ and R² are substituents, and n, W, X and Y are defined within.

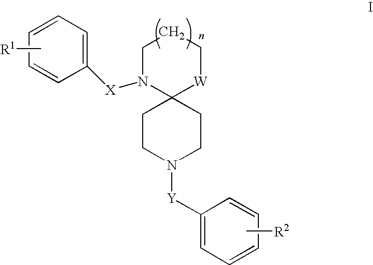

### CROSS-REFERENCE TO RELATED APPLICATION

This applications claims priority from application Ser. No. 12/263,257 that was filed on Oct. 31, 2008, and whose disclosures are incorporated herein by reference.

### TECHNICAL FIELD

This invention contemplates a composition and related method for providing opioid-strength analgesia while minimizing analgesic tolerance, physical dependence and addiction. More particularly, a composition and method are described that utilize a small molecule to inhibit the interaction of the mu opioid receptor with filamin A, either by binding to filamin A itself or by mimicking filamin A's binding to the mu opioid receptor. Preferably, the composition prevents this mu opioid receptor-filamin A interaction and also functions as a mu opioid receptor agonist. Most preferably, the composition binds filamin A with picomolar or sub-picomolar affinity.

### BACKGROUND OF THE INVENTION

Opiates are powerful analgesics (agents used for the treatment of pain), but their use is hampered by non-trivial side effects, tolerance to the analgesic effects, physical dependence resulting in withdrawal effects, and by concerns surrounding the possibility of addiction.

Opiates produce analgesia by activation of opioid receptors that belong to the rhodopsin-like superfamily of G protein-coupled receptors (GPCRs).

Adaptive responses of opioid receptors contribute to the development of analgesic tolerance and physical dependence, and possibly also to components of opioid addiction.

Opiates produce analgesia by activation of mu (μ) opioid receptor-linked inhibitory G protein signaling cascades and related ion channel interactions that suppress cellular activities by hyperpolarization. The μ opioid receptor (MOR) preferentially couples to pertussis toxin-sensitive G proteins, Gαi/o (inhibitory/other), and inhibits the adenylyl cyclase/cAMP pathway (Laugwitz et al., 1993 Neuron 10:233-242; Connor et al., 1999 Clin Exp Pharmacol Physiol 26:493-499). The analgesic effects of MOR activation have been predominantly attributed to the Gβy dimer released from the Gαi/o protein, which activates G protein activated inwardly rectifying potassium (GIRK) channels (Ikeda et al., 2000 Neurosci Res 38:113-116) and inhibits voltage-dependent calcium channels (VDCCs) (Saegusa et al., 2000 Proc Natl Acad Sci USA 97:6132-6137), thereby suppressing cellular activities by hyperpolarization.

Adenylyl cyclase inhibition can also contribute to opioid analgesia, or importantly, its activation can contribute to analgesic tolerance. The role of adenylyl cyclase inhibition or activation in opioid analgesia and analgesic tolerance, respectively, is evidenced by overexpression of adenylyl cyclase type 7 in the CNS of mice leading to more rapid tolerance to morphine (Yoshimura et al., 2000 Mol Pharmacol 58:1011-1016). Additionally, adenylyl cyclase activitation has been suggested to elicit analgesic tolerance or tolerance-associated hyperalgesia (Wang et al., 1997 J Neurochem 68:248-254). Although the superactivation of adenylyl cyclase after chronic opioid administration is more often viewed as a hallmark of opioid dependence than as a mediator of tolerance (Nestler, 2001 Am J Addict 10:201-217), both are consequences of chronic opioid administration, and tolerance often worsens dependence. Chronic pain patients who have escalated their opioid dose over time often experience more withdrawal than patients on a constant dose.

An important but underemphasized cellular consequence of chronic opioid treatment is MOR excitatory signaling, by activation of adenylyl cyclase, in place of the usual inhibitory signaling or inhibition of adenylyl cyclase (Crain et al., 1992 Brain Res 575:13-24; Crain et al., 2000 Pain 84:121-131; Gintzler et al., 2001 Mol Neurobiol 21:21-33; Wang et al., 2005 Neuroscience 135:247-261). This change in signaling is caused by a switch in G protein coupling from Gi/o to Gs (Wang et al., 2005 Neuroscience 135:247-261) and may be a result of the decreased efficiency of coupling to the native G proteins, the usual index of desensitization (Sim et al., 1996 J Neurosci 16:2684-2692) and still commonly considered the reason for analgesic tolerance.

The chronic opioid-induced MOR-G protein coupling switch (Wang et al 2005 Neuroscience 135:247-261; Chakrabarti et al., 2005 Mol Brain Res 135:217-224) is accompanied by stimulation of adenylyl cyclase II and IV by MOR-associated Gβγ dimers (Chakrabarti et al., 1998 Mol Pharmacol 54:655-662; Wang et al., 2005 Neuroscience 135:247-261). The interaction of the Gβγ dimer with adenylyl cyclase had previously been postulated to be the sole signaling change underlying the excitatory effects of opiates (Gintzler et al., 2001 Mol Neurobiol 21:21-33). It has further been shown that the Gβγ that interacts with adenylyl cyclases originates from the Gs protein coupling to MOR and not from the Gi/o proteins native to MOR (Wang et al., 2006 J Neurobiol 66:1302-1310).

Thus, MORs are normally inhibitory G protein-coupled receptors that couple to Gi or Go proteins to inhibit adenylyl cyclase and decrease production of the second messenger cAMP, as well as to suppress cellular activities via ion channel-mediated hyperpolarization. Opioid analgesic tolerance and dependence are also associated with that switch in G protein coupling by MOR from Gi/o to Gs (Wang et al., 2005 Neuroscience 135:247-261). This switch results in activation of adenylyl cyclase that provides essentially opposite, stimulatory, effects on the cell.

Controlling this switch in G protein coupling by MOR is the scaffolding protein filamin A, and compounds that bind a particular segment of filamin A with high affinity, like naloxone (NLX) and naltrexone (NTX), can prevent this switch (Wang et al, 2008 PLoS One 3:e1554) and the associated analgesic tolerance and dependence(Wang et al., 2005 Neuroscience 135:247-261). This switch in G protein coupling also occurs acutely, though transiently, and is potentially linked to the acute rewarding or addictive effects of opioid drugs, through CREB activation as a result of increased cAMP accumulation (Wang et al., 2009 PLoS ONE 4(1):e4282).

Ultra-low-dose NLX or NTX have been shown to enhance opioid analgesia, minimize opioid tolerance and dependence (Crain et al., 1995 Proc Natl Acad Sci USA 92:10540-10544; Powell et al. 2002. JPET 300:588-596), as well as to attenuate the addictive properties of opioids (Leri et al., 2005 Pharmacol Biochem Behav 82:252-262; Olmstead et al., 2005 Psychopharmacology 181:576-581). An ultra-low dose of opioid antagonist was an amount initially based on in vitro studies of nociceptive dorsal root ganglion neurons and on in vivo mouse studies, wherein the amount of the excitatory opioid receptor antagonist administered is about 1000- to about 10,000,000-fold less, preferably about 10,000- to about 1,000,000-fold less than the amount of opioid agonist administered. It has long been hypothesized that ultra-low-dose opioid antagonists enhance analgesia and alleviate tolerance/dependence by blocking the excitatory signaling opioid receptors that underlie opioid tolerance and hyperalgesia (Crain et al., 2000 Pain 84:121-131). Later research has shown that the attenuation of opioid analgesic tolerance, dependence and addictive properties by ultra-low-dose, defined herein, naloxone or naltrexone, occurs by preventing the MOR-Gs coupling that results from chronic opiate administration (Wang et al., 2005 Neuroscience 135:247-261), and that the prevention of MOR-Gs coupling is a result of NLX or NTX binding to filamin A at approximately 4 picomolar affinity (Wang et al, 2008 PLoS One 3:e1554).

Found in all cells of the brain, CREB is a transcription factor implicated in addiction as well as learning and memory and several other experience-dependent, adaptive (or maladaptive) behaviors (Carlezon et al., 2005 Trends Neurosci 28:436-445). In general, CREB is inhibited by acute opioid treatment, an effect that is completely attenuated by chronic opioid treatment, and activated during opioid withdrawal (Guitart et al., 1992 J Neurochem 58:1168-1171). However, a regional mapping study showed that opioid withdrawal activates CREB in locus coeruleus, nucleus accumbens and amygdala but inhibits CREB in lateral ventral tegemental area and dorsal raphe nucleus (Shaw-Luthman et al., 2002 J Neurosci 22:3663-3672).

In the striatum, CREB activation has been viewed as a homeostatic adaptation, attenuating the acute rewarding effects of drugs (Nestler, 2001 Am J Addict 10:201-217; Nestler, 2004 Neuropharmacology 47:24-32). This view is supported by nucleus accumbens overexpression of CREB or a dominant-negative mutant respectively reducing or increasing the rewarding effects of opioids in the conditioned place preference test (Barot et al., 2002 Proc Natl Acad Sci USA 99:11435-11440). In conflict with this view, however, reducing nucleus accumbens CREB via antisense attenuated cocaine reinforcement as assessed in self-administration (Choi et al., 2006 Neuroscience 137:373-383). Clearly, CREB activation is implicated in addiction, but whether it directly contributes to the acute rewarding effects of drugs or initiates a homeostatic regulation thereof appears less clear.

The several-fold increase in pS¹³³CREB reported by Wang et al., 2009 PLoS ONE 4(1):e4282 following acute, high-dose morphine may indicate acute dependence rather than acute rewarding effects. However, the transient nature of the MOR-Gs coupling correlating with this CREB activation suggests otherwise. In fact, the correlation of pS¹³³CREB with the Gs coupling by MOR following this acute high-dose morphine exposure, as well as the similar treatment effects on both, suggest that this alternative signaling mode of MOR can contribute to the acute rewarding or addictive effects of opioids. This counterintuitive notion can explain the apparent paradox that ultra-low-dose NTX, while enhancing the analgesic effects of opioids, decreases the acute rewarding or addictive properties of morphine or oxycodone as measured in conditioned place preference or self-administration and reinstatement paradigms.

In considering analgesic tolerance, opioid dependence, and opioid addiction together as adaptive regulations to continued opioid exposure, a treatment that prevents MOR's signaling adaptation of switching its G protein partner can logically attenuate these seemingly divergent behavioral consequences of chronic opioid exposure. However, the acute rewarding effects of opioids are not completely blocked by ultra-low-dose opioid antagonists, suggesting that a MOR-Gs coupling can only partially contribute to the addictive or rewarding effects.

Even though ultra-low-dose NTX blocks the conditioned place preference to oxycodone or morphine (Olmstead et al., 2005 Psychopharmacology 181:576-581), its co-self-administration only reduces the rewarding potency of these opioids but does not abolish self-administration outright (Leri et al., 2005 Pharmacol Biochem Behav 82:252-262). Nevertheless, it is possible that a direct stimulatory effect on VTA neurons, as opposed to the proposed disinhibition via inhibition of GABA interneurons (Spanagel et al., 1993 Proc Natl Acad Sci USA 89:2046-2050), can play some role in opioid reward. Finally, a MOR-Gs coupling mediation of reward, increasing with increasing drug exposure, is in keeping with current theories that the escalation of drug use signifying drug dependence can not indicate a “tolerance” to rewarding effects but instead a sensitization to rewarding effects (Zernig et al., 2007 Pharmacology 80:65-119).

The above results reported in Wang et al., 2009 PLoS ONE 4(1):e4282 demonstrated that acute, high-dose morphine causes an immediate but transient switch in G protein coupling by MOR from Go to Gs similar to the persistent switch caused by chronic morphine. Ultra-low-dose NLX or NTX prevented this switch and attenuated the chronic morphine-induced coupling switch by MOR. The transient nature of this acute altered coupling suggests the receptor eventually recovers and couples to its native G protein.

With chronic opioid exposure, the receptor can lose the ability to recover and continue to couple to Gs, activating the adenylyl cyclase/cAMP pathway, upregulating protein kinase A, and phosphorylating CREB as one downstream effector example. The persistently elevated phosphorylated CREB can then shape the expression of responsive genes including those closely related to drug addiction and tolerance. Importantly, the equivalent blockade of Gs coupling and pS¹³³CREB by the pentapeptide binding site of naloxone (NLX) and naltrexone (NTX) on FLNA further elucidates the mechanism of action of ultra-low-dose NLX and NTX in their varied effects.

These data further strengthen a mechanistic basis for MOR-Gs coupling through the interaction between FLNA and MOR and that disrupting this interaction, either by NLX/NTX binding to FLNA or via a FLNA peptide decoy for MOR, the altered coupling is prevented, resulting in attenuation of tolerance, dependence and addictive properties associated with opioid drugs.

The combination of ultra-low-dose opioid antagonists with opioid agonists formulated together in one medication has been shown to alleviate many of these undesirable aspects of opioid therapy (Burns, 2005 Recent Developments in Pain Research 115-136, ISBN:81-308-0012-8). This approach shows promise for an improvement in analgesic efficacy, and animal data suggests reduced addictive potential. The identification of the cellular target of ultra-low-dose NLX or NTX in their inhibition of mu opioid receptor-Gs coupling as a pentapeptide segment of filamin A (Wang et al., 2008 PLoS ONE 3(2):e1554) has led to development of assays to screen against this target to create a new generation of pain therapeutics that can provide long-lasting analgesia with minimal tolerance, dependence and addictive properties. Importantly, the non-opioid cellular target of ultra-low-dose NLX or NTX, FLNA, provides potential for developing either a therapeutic combination of which one component is not required to be ultra-low-dose, or a single-entity novel analgesic.

The present invention identifies a compound that is similar to or more active than DAMGO in activating the mu (μ) opioid receptor (MOR), and that also binds to filamin A (FLNA; the high-affinity binding site of naloxone [NLX] and naltrexone [NTX]), thereby preventing the Gi/o-to-Gs coupling switch of MOR to attenuate opioid tolerance, dependence and addiction.

### BRIEF SUMMARY OF THE INVENTION The present invention contemplates an analgesic compound and a method of reducing pain in a host mammal in need thereof by administering a composition containing such a compound. A contemplated compound corresponds in structure to Formula I

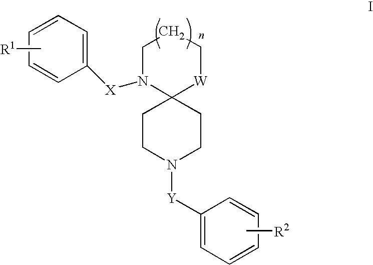

In Formula I, X and Y are the same or different and are SO₂, C(O) or NHC(O); W is NR⁷ or O, where R⁷ is H, C₁-C₆ hydrocarbyl, or C₁-C₇ hydrocarboyl(acyl); n is zero or one; and R¹ and R² are the same or different and are selected from the group consisting of H, C₁-C₆ hydrocarbyl, C₁-C₆ hydrocarbyloxy, trifluoromethyl, trifluoromethoxy, C₁-C₇ hydrocarboyl(acyl), C₁-C₆ hydrocarbylsulfonyl, halogen, nitro, phenyl, cyano, carboxyl, C₁-C₇ hydrocarbyl carboxylate, carboxamide wherein the amido nitrogen has the formula NR³ R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, and NR⁵R⁶ wherein R⁵ and R⁶ are the same or different and are H, C₁-C₄ hydrocarbyl, C₁-C₄ acyl, C₁-C₄ hydrocarbylsulfonyl, or R⁵ and R⁶ together with the, depicted nitrogen form a 5-7-membered ring that optionally contains 1 or 2 additional hetero atoms that independently are nitrogen, oxygen or sulfur. However, in a compound of Formula I, R¹ and R² are not both methoxy when X and Y are both SO₂, W is O and n is zero.

In preferred embodiments, X and Y are both SO₂. In those and other embodiments, W is preferably O. It is also preferred that n be zero.

There are several independent and separate preferences regarding the substituent R groups. Thus, R¹ and R² are preferably the same; R¹ and R² are preferably located at the same relative position in their respective rings, and R¹ and R² preferably also have a Hammett sigma value for a para-position substituent that is greater than −0.2, and more preferably, a Hammett sigma value for a para-position substituent that is positive (greater than zero).

A pharmaceutical composition is also contemplated. That composition comprises an above compound of Formula I or a compound of Formula I in which R¹ and R² are both methoxy when X and Y are both SO₂, W is O and n is zero dissolved or dispersed in a physiologically tolerable carrier. The compound is present in an effective analgesic amount. The composition is preferably in solid form as in a tablet of capsule.

A method of reducing pain in a host mammal in need thereof is also contemplated. That method comprises administering to that host mammal a pharmaceutical composition as disclosed above. The host mammal for such a method is selected from the group consisting of a primate, a laboratory rodent, a companion animal, and a food animal. A composition can be administered a plurality of times over a period of days, as well as administered a plurality of times in one day. That administration can be perorally or parenteral.

The present invention has several benefits and advantages.

One benefit is that analgesia can be provided at morphine-like potency by a compound that does not have a narcotic structure.

An advantage of the invention is that analgesia can be provided by administration of acontemplated composition either perorally or parenterally.

A further benefit of the invention is that as indicated by the initial data, a contemplated compound provides the analgesic effects characteristic of opioid drugs but does not cause analgesic tolerance or dependence.

Another advantage of the invention as also indicated by the initial data is that a contemplated compound provides the analgesic effects characteristic of opioid drugs and does not have the addictive potential of opioid drugs.

Still further benefits and advantages will be apparent to a skilled worker from the description that follows.

### Abbreviations and Short Forms

The following abbreviations and short forms are used in this specification.

“MOR” means μ-opioid receptor

“FLNA” means filamin A

“NIX” means naloxone

“NTX” means naltrexone

“Gαi/o” means G protein alpha subunit-inhibitory/other conformation, inhibits adenylyl cyclase

“Gαs” means G protein alpha subunit-stimulatory conformation stimulates adenylyl cyclase

“Gβγ” means G protein beta gamma subunit

“cAMP” means cyclic adenosine monophosphate

“CREB” means cAMP Response Element Binding protein

“IgG” means Immunoglobulin G

### Definitions

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

As used herein, the term “hydrocarbyl” is a short hand term to include straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrocarbyl groups, are referred to herein as aryl groups, substituents, moieties or radicals, as discussed hereinafter. An aralkyl group such as benzyl or phenethyl is deemed a hydrocarbyl group. Where a specific aliphatic hydrocarbyl substituent group is intended, that group is recited; i.e., C₁-C₄ alkyl, methyl or dodecenyl. Exemplary hydrocarbyl groups contain a chain of 1 to about 12 carbon atoms, and preferably 1 to about 7 carbon atoms, and more preferably 1 to 4 carbon atoms of an alkyl group.

A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Examples of alkyl radicals include methyl, ethyl, n-propyl, isopropyl, n-butyl, isobutyl, sec-butyl, tert-butyl, pentyl, iso-amyl, hexyl, octyl, decyl, dodecyl and the like. Examples of suitable alkenyl radicals include ethenyl(vinyl), 2-propenyl, 3-propenyl, 1,4-pentadienyl, 1,4-butadienyl, 1-butenyl, 2-butenyl, 3-butenyl, decenyl and the like. Examples of alkynyl radicals include ethynyl, 2-propynyl, 3-propynyl, decynyl, 1-butynyl, 2-butynyl, 3-butynyl, and the like.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, and cyclohexenyloxy groups. On the other hand, a hydrocarbyl group containing a —C(O)O— functionality is referred to as a hydrocarboyl(acyl) or hydrocarboyloxy group inasmuch as there is no ambiguity. Exemplary hydrocarboyl and hydrocarboyloxy groups include acyl and acyloxy groups, respectively, such as acetyl and acetoxy, acryloyl and acryloyloxy.

A “carboxyl” substituent is a —C(O)OH group. A C₁-C₆ hydrocarbyl carboxylate is a C₁-C₆ hydrocarbyl ester of a carboxyl group. A carboxamide is a —C(O)NR³R⁴ substituent, where the R groups are defined elsewhere. Illustrative R³ and R⁴ groups that together with the depicted nitrogen of a carboxamide form a 5-7-membered ring that optionally contains 1 or 2 additional hetero atoms that independently are nitrogen, oxygen or sulfur, include morpholinyl, piperazinyl, oxathiazolyl, 1,2,3-triazolyl, 1,2,4-triazolyl, pyrazolyl, 1,2,4-oxadiazinyl and azepinyl groups.

As a skilled worker will understand, a substituent that cannot exist such as a C₁ alkenyl or alkynyl group is not intended to be encompassed by the word “hydrocarbyl”, although such substituents with two or more carbon atoms are intended.

The term “aryl”, alone or in combination, means a phenyl or naphthyl radical that optionally carries one or more substituents selected from hydrocarbyl, hydrocarbyloxy, halogen, hydroxy, amino, nitro and the like, such as phenyl, p-tolyl, 4-methoxyphenyl, 4-(tent-butoxy)phenyl, 4-fluorophenyl, 4-chlorophenyl, 4-hydroxyphenyl, and the like. The term “arylhydrocarbyl”, alone or in combination, means a hydrocarbyl radical as defined above in which one hydrogen atom is replaced by an aryl radical as defined above, such as benzyl, 2-phenylethyl and the like. The term “arylhydrocarbyloxycarbonyl”, alone or in combination, means a radical of the formula —C(O)—O— arylhydrocarbyl in which the term “arylhydrocarbyl” has the significance given above. An example of an arylhydrocarbyloxycarbonyl radical is benzyloxycarbonyl. The term “aryloxy” means a radical of the formula aryl-O— in which the term aryl has the significance given above. The term “aromatic ring” in combinations such as substituted-aromatic ring sulfonamide, substituted-aromatic ring sulfinamide or substituted-aromatic ring sulfenamide means aryl or heteroaryl as defined above.

As used herein, the term “binds” refers to the adherence of molecules to one another, such as, but not limited to, peptides or small molecules such as the compounds disclosed herein, and opioid antagonists, such as naloxone or naltrexone.

As used herein, the term “selectively binds” refers to binding as a distinct activity. Examples of such distinct activities include the independent binding to filamin A or a filamin A binding peptide, and the binding of a compound discussed above to a p opioid receptor.

As used herein, the term “FLNA-binding compound” refers to a compound that binds to the scaffolding protein filamin A, or more preferably to a polypeptide comprising residues -Val-Ala-Lys-Gly-Leu- (SEQ ID NO:1) of the FLNA sequence that correspond to amino acid residue positions 2561-2565 of the FLNA protein sequence as noted in the sequence provided at the web address: UniProtKB/Swiss-Prot entry P21333, FLNA-HUMAN, Filamin-A protein sequence. A FLNA-binding compound can inhibit the MOR-Gs coupling caused by agonist stimulation of the μ opioid receptor via interactions with filamin A, preferably in the 24^{th} repeat region. When co-administered with an opioid agonist, a FLNA-binding compound can enhance the analgesic effects and improve the treatment of pain.

As used herein, the term “candidate FLNA-binding compound” refers to a substance to be screened as a potential FLNA-binding compound. In preferred instances a FLNA-binding compound is also an opioid agonist. Additionally, a FLNA-binding compound can function in a combinatory manner similar to the combination of an opioid agonist and ultra-low-dose antagonist, wherein both FLNA and the mu-opioid receptor are targeted by a single entity.

As used herein, the term “opioid receptor” refers to a G protein coupled receptor, located in the central nervous system that interacts with opioids. More specifically, the μ opioid receptor is activated by morphine causing analgesia, sedation, nausea, and many other side effects known to one of ordinary skill in the art.

As used herein, the term “opioid agonist” refers to a substance that upon binding to an opioid receptor can stimulate the receptor, induce G protein coupling and trigger a physiological response. More specifically, an opioid agonist is a morphine-like substance that interacts with MOR to produce analgesia.

As used herein, the term “opioid antagonist” refers to a substance that upon binding to an opioid receptor inhibits the function of an opioid agonist by interfering with the binding of the opioid agonist to the receptor.

As used herein an “analgesia effective amount” refers to an amount sufficient to provide analgesia or pain reduction to a recipient host.

As used herein the term “ultra-low-dose” or “ultra-low amount” refers to an amount of compound that when given in combination with an opioid agonist is sufficient to enhance the analgesic potency of the opioid agonist. More specifically, the ultra-low-dose of an opioid antagonist admixed with an opioid agonist in mammalian cells is an amount about 1000- to about 10,000,000-fold less, and preferably between about 10,000- and to about 1,000,000-fold less than the amount of opioid agonist.

As used herein an “FLNA-binding effective amount” refers to an amount sufficient to perform the functions described herein, such as inhibition of MOR-Gs coupling, prevention of the cAMP desensitization measure, inhibition of CREB S¹³³ phosphorylation and inhibition of any other cellular indices of opioid tolerance and dependence, which functions can also be ascribed to ultra-low-doses of certain opioid antagonists such as naloxone or naltrexone. When a polypeptide or FLNA-binding compound of the invention interacts with FLNA, an FLNA-binding effective amount can be an ultra-low amount or an amount higher than an ultra-low-dose as the polypeptide or FLNA-binding compound will not antagonize the opioid receptor and compete with the agonist, as occurs with known opioid antagonists such as naloxone or naltrexone in amounts greater than ultra-low-doses. More preferably, when a polypeptide or VAKGL-binding compound of the present invention both interacts with FLNA and is an agonist of the mu opioid receptor, an FLNA-binding effective amount is an amount higher than an ultra-low-dose and is a sufficient amount to activate the mu opioid receptor.

As used herein the phrase “determining inhibition of the interaction of a mu opioid receptor with a Gs protein” refers to monitoring the cellular index of opioid tolerance and dependence caused by chronic or high-dose administration of opioid agonists to mammalian cells. More specifically, the mu opioid receptor-Gs coupling response can be identified by measuring the presence of the Gas (stimulatory) subunit, the interaction of MOR with the G protein complexes and formation of Gs-MOR coupling, the interaction of the Gβγ protein with adenylyl cyclase types II and IV, loss of inhibition or outright enhancement of cAMP accumulation, and the activation of CREB via phosphorylation of S¹³³.

As used herein the term “naloxone/naltrexone positive control” refers to a positive control method comprising steps discussed in a method embodiment, wherein the candidate FLNA-binding compound is a known opioid antagonist administered in an ultra-low amount, preferably naloxone or naltrexone.

As used herein the term “FLNA-binding compound negative control” refers to a negative control method comprising steps discussed in a method embodiment, wherein the candidate FLNA-binding compound is absent and the method is carried out in the presence of only opioid agonist.

As used herein the term “pharmacophore” is not meant to imply any pharmacological activity. The term refers to chemical features and their distribution in three-dimensional space that constitutes and epitomizes the preferred requirements for molecular interaction with a receptor (U.S. Pat. No. 6,034,066).

### DETAILED DESCRIPTION OF THE INVENTION

It should be understood that the present disclosure is to be considered as an exemplification of the present invention, and is not intended to limit the invention to the specific embodiments illustrated. It should be further understood that the title of this section of this application (“Detailed Description of the Invention”) relates to a requirement of the United States Patent Office, and should not be found to limit the subject matter disclosed herein.

The present invention contemplates a compound that binds to FLNA and also stimulates the μ opioid receptor (MOR), and method of its use to provide analgesia. A contemplated compound can inhibit MOR-Gs coupling through interactions with FLNA and/or the μ opioid receptor (MOR).

In another aspect of the present invention, a contemplated compound prevents the morphine-induced Gs protein coupling by MOR. That prevention of MOR-Gs coupling is believed to occur by preventing the interaction of filamin A and MOR. Downstream effects of preventing the MOR-Gs coupling include inhibition of cAMP accumulation and of cAMP Response Element Binding protein (CREB) activation in a manner resembling the activity of ultra-low-dose opioid antagonists naloxone and naltrexone.

In another aspect of the present invention, a FLNA-binding compound prevents the MOR-Gs coupling while itself activating MOR.

The data collected in organotypic striatal slice cultures demonstrate that after 7 days of twice daily 1-hour exposures to oxycodone, mu opioid receptors in striatum switch from Go to Gs coupling (compare vehicle to oxycodone conditions). In contrast, a compound contemplated herein did not cause a switch to Gs coupling despite its ability to stimulate mu opioid receptors as previously assessed by GTPγS binding that is blocked by beta-funaltrexamine, a specific mu opioid receptor antagonist. These data imply that these compounds provide the analgesic effects characteristic of opioid drugs but do not cause analgesic tolerance or dependence, and do not have the addictive potential of opioid drugs.

A compound contemplated by the present invention binds to an above-defined FLNA polypeptide as well as stimulates the μ opioid receptor (MOR). A contemplated compound corresponds in structure to Formula I

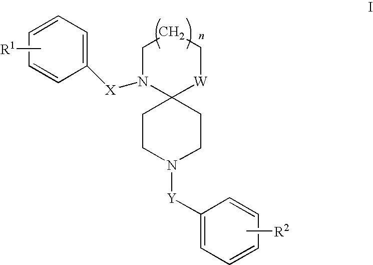

wherein

X and Y are the same or different and are SO₂, C(O) or NHC(O);

W is NR⁷ or O, where R⁷ is H, C₁-C₆ hydrocarbyl, or C₁-C₇ hydrocarboyl(acyl);

n is zero or one; and

R¹ and R² are the same or different and are selected from the group consisting of H, C₁-C₆ hydrocarbyl, C₁-C₆ hydrocarbyloxy, trifluoromethyl, trifluoromethoxy, C₁-C₇ hydrocarboyl(acyl), C₁-C₆ hydrocarbylsulfonyl, halogen, nitro, phenyl, cyano, carboxyl, C₁-C₇ hydrocarbyl carboxylate, carboxamide wherein the amido nitrogen has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, and NR⁵R⁶ wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur;

with the proviso that R¹ and R² are not both methoxy when X and Y are both SO₂, W is O and n is zero.

Thus, X and Y can form a sulfonamide, a carboxamide or a urea linkage from the phenyl ring to a depicted nitrogen atom of the central spiro ring. A compound having a central ring that is a spiro 6,6-ring system or a spiro 5,6-ring system, along with one nitrogen and one oxygen or two nitrogens is contemplated. Illustrative central rings are shown below where wavy lines are used to indicate the presence of covalent bonds to other entities, and where R⁷ is defined above.

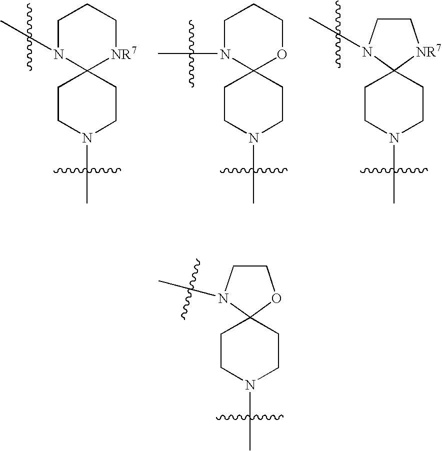

In preferred practice, n is zero, so the central ring is a spiro 5,6-ring system. It is separately preferred that W be O. A compound in which X and Y are the same are preferred. It is also separately preferred that X and Y both be SO₂ (sulfonyl).

A particularly preferred compound of Formula I that embodies the above separate preferences is a compound of Formula II

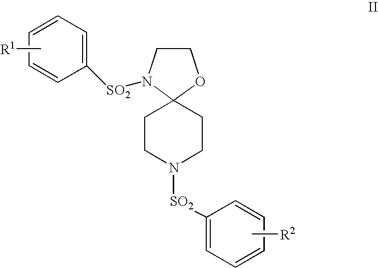

where R¹ and R² are as described previously.

There are several independent and separate preferences regarding the substituent R groups. Thus, R¹ and R² are preferably the same. R¹ and R² are also preferably located at the same relative position in their respective rings. Thus, if R¹ is 4-cyano, R² is also 4-cyano.

R¹ and R² preferably also have a Hammett sigma value for a para-position substituent that is greater than −0.2, and more preferably, a Hammett sigma value for a para-position substituent that is positive (greater than zero). Hammett sigma values are well known in organic chemistry and those values for para-position substituents reflect both electron donation or withdrawal via an inductive effect, but also are understood to reflect a resonance effect. See, for example, U.S. Pat. No. 7,473,477, U.S. Pat. No. 5,811,521, U.S. Pat. No. 4,746,651, and U.S. Pat. No. 4,548,905. A list of Hammett sigma values can be found in J. Hine, Physical Organic Chemistry, 2^{nd} ed., McGraw-Hill Book Co., Inc., New York page 87 (1962) and at the web site: wiredchemist.com/chemistry/data/hammett sigma constants.

Most of the compounds assayed having substituents with Hammett sigma values for a para-position substituent that are greater than −0.2 are more active than are assayed compounds with Hammett sigma values for a para-position substituent that are less than −0.2 (more negative). The most active compounds have substituents whose Hammett sigma values for a para-position substituent are positive; i.e., greater than zero. It is also noted that preferred R¹ and R² substituent groups do not themselves provide a positive or negative charge to a compound at a pH value of about 7.2-7.4.

A particularly preferred compound of Formula II that embodies the above separate preferences is selected from the group consisting of:

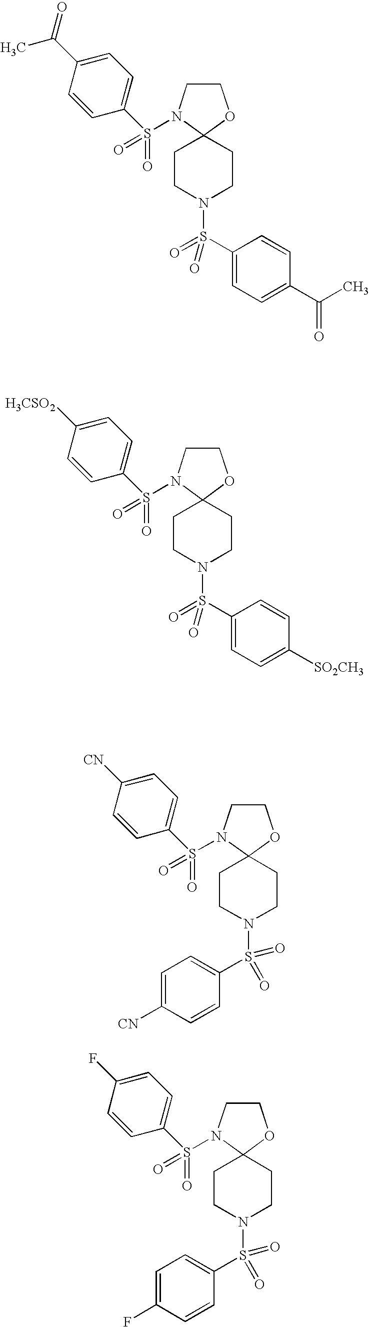

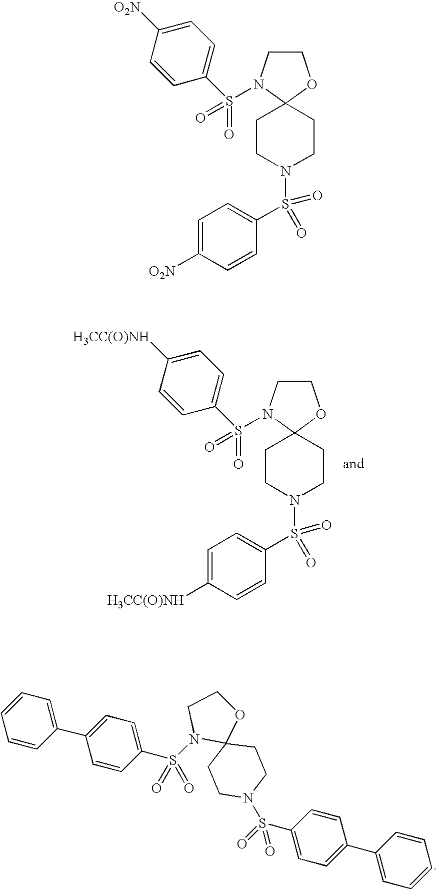

In other embodiments, a particularly preferred compound of Formula I is a compound of Formula III

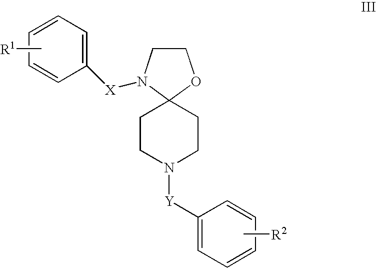

wherein

X and Y are both CO, or X is SO₂ and Y is CO; and

R¹ and R² are the same and are selected from the group consisting of trifluoromethyl, C₁-C₆ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

A particular compound of Formula III is

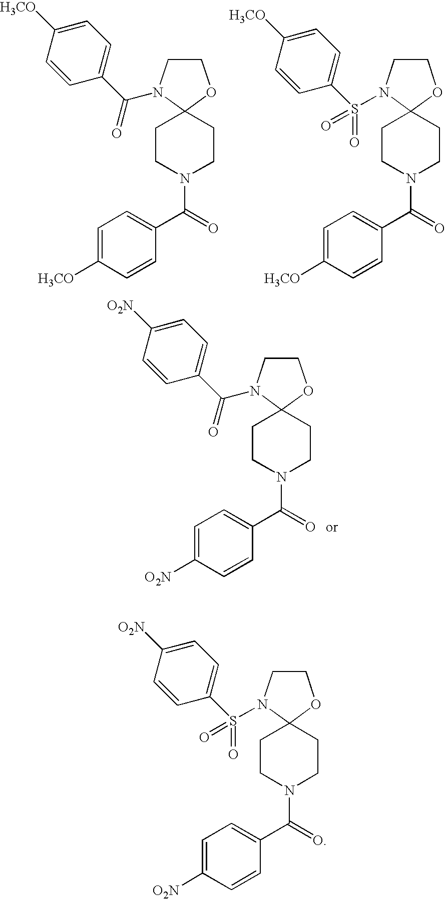

The present invention also contemplates a method of treatment to reduce pain in a treated mammal. A compound of Formulas I, II and III present in an analgesic effective amount dissolved or dispersed in a physiologically tolerable diluent can and preferably is used in such a treatment. However, a compound of Formula IV in an analgesic effective amount dissolved or dispersed in a physiologically tolerable diluent is also contemplated. In Formula IV,

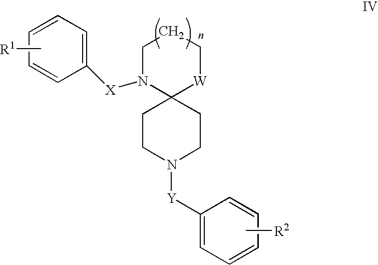

X and Y are the same or different and are SO₂, C(O) or NHC(O);

W is NR⁷ or O, where R⁷ is H, C₁-C₆ hydrocarbyl, or C₁-C₇ acyl; and

R¹ and R² are the same or different and are selected from the group consisting of H, C₁-C₆ hydrocarbyl, C₁-C₆ hydrocarbyloxy, trifluoromethyl, trifluoromethoxy, C₁-C₇ acyl, C₁-C₆ hydrocarbylsulfonyl, halogen, nitro, phenyl, cyano, carboxyl, C₁-C₆ hydrocarbyl carboxylate, carboxamide wherein the amido nitrogen has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, and NR⁵R⁶ wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

Thus, a compound of Formula IV encompasses compounds in addition to those of Formula I. In particular, R¹ and R² substituents of a compound of Formula IV include C₁-C₆ hydrocarbyloxy and amino substituents NR⁵R⁶. These R¹ and R² groups have Hammett sigma values for the para-position that are less than −0.2. For example, the Hine text, above, lists appropriate para-position sigma values for methoxy and ethoxy groups as −0.268 and −0.24, respectively. The para-position sigma value for an unsubstituted amine is −0.66, whereas a dimethylamino group has a reported para-position sigma value of −0.83.

Aside from the inclusion of additional R¹ and R² groups, the preferences discussed above for a compound of Formula I also apply to a compound of Formula IV. Thus, n is preferably zero, and W is preferably O. X and Y are preferably the same and are SO₂.

In another aspect, a contemplated compound is selected in part using a method for determining the ability of a candidate FLNA-binding compound, other than naloxone or naltrexone, to inhibit the interaction of the mu opioid receptor with filamin A (FLNA) and thereby prevent the mu opioid receptor from coupling to Gs proteins (Gs). That method comprises the steps of: (a) admixing the candidate FLNA-binding compound (alone if such FLNA-binding compound also stimulates MOR or with a MOR agonist otherwise) with mammalian cells that contain the mu opioid receptor and FLNA in their native conformations and relative orientations, the opioid agonist being present in an agonist effective amount and/or being administered in a repeated, chronic manner the FLNA-binding compound being present in an FLNA-binding effective amount; and (b) determining inhibition of the interaction of the mu opioid receptor with the G protein by analysis of the presence or the absence of the Gas subunit of Gs protein, wherein the absence of the Gas subunit indicates inhibition of the interaction of the mu opioid receptor with the Gs protein.

In one aspect, the analysis of Gs protein coupling by the mu opioid receptor and downstream effects elicited by admixing mammalian cells with a before-defined compound can be conducted by any one or more of several methods such as for example co-immunoprecipitation of Gα proteins with MOR, Western blot detection of MOR in immunoprecipitates, and densitometric quantification of Western blots.

### Pharmaceutical Composition

A pharmaceutical composition is contemplated that contains an analgesia effective amount of a compound of Formula I, Formula II, Formula III, or Formula IV dissolved or dispersed in a physiologically tolerable carrier. Such a composition can be administered to mammalian cells in vitro as in a cell culture, or in vivo as in a living, host mammal in need.

A contemplated composition is typically administered a plurality of times over a period of days. More usually, a contemplated composition is administered a plurality of times in one day.

As is seen from the data that follow, a contemplated compound is active in the assays studies at micromolar amounts. In the laboratory mouse tail flick test, orally administered morphine exhibited an A₅₀ value of 61.8 (52.4-72.9) mg/kg, and a mean maximum antinoniception amount of about 43% at 56 mg/kg at about 20 minutes. Orally administered compound C0011 (see the Table of Correspondence hereinafter for a correlation of structures and compound numbers) exhibited a mean maximum antinoniception amount of about 70% at 56 mg/kg at about 10-20 minutes, whereas orally administered compound C0009 exhibited a mean maximum antinoniception amount of about 50% at 56 mg/kg at about 10 minutes, and compound C0022 exhibited a mean maximum antinoniception amount of about 40% at 56 mg/kg at about 30 minutes. It is thus seen that the contemplated compounds are quite active and potent, and that a skilled worker can readily determine an appropriate dosage level to achieve a desired amount of pain reduction, particularly in view of the relative activity of a contemplated compound compared to orally administered morphine.

A contemplated pharmaceutical composition can be administered orally (perorally), parenterally, by inhalation spray in a formulation containing conventional nontoxic pharmaceutically acceptable carriers, adjuvants, and vehicles as desired. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline. Liquid pharmaceutical compositions include, for example, solutions suitable for parenteral administration. Sterile water solutions of an active component or sterile solution of the active component in solvents comprising water, ethanol, or propylene glycol are examples of liquid compositions suitable for parenteral administration.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

A mammal in need of treatment and to which a pharmaceutical composition containing a contemplated compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

Where in vitro mammalian cell contact is contemplated, a CNS tissue culture of cells from an illustrative mammal is often utilized, as is illustrated hereinafter. In addition, a non-CNS tissue preparation that contains opioid receptors such as guinea pig ileumcan also be used.

Preferably, the pharmaceutical composition is in unit dosage form. In such form, the composition is divided into unit doses containing appropriate quantities of the active urea. The unit dosage form can be a packaged preparation, the package containing discrete quantities of the preparation, for example, in vials or ampules.

### Examples

The present invention is described in the following examples which are set forth to aid in the understanding of the invention, and should not be construed to limit in any way the invention as defined in the claims which follow thereafter.

The experiments described herein were carried out on organotypic striatal slices from male Sprague Dawley rats (200 to 250 g) purchased from Taconic (Germantown, N.Y.). Rats were housed two per cage and maintained on a regular 12-hour light/dark cycle in a climate-controlled room with food and water available ad libitum and sacrificed by rapid decapitation. All data are presented as mean±standard error of the mean. Treatment effects were evaluated by two-way ANOVA followed by Newman-Keul's test for multiple comparisons. Two-tailed Student's t test was used for post hoc pairwise comparisons. The threshold for significance was p<0.05.

The following Table of Correspondence shows the structures of the compounds discussed herein and their identifying numbers.

Table of Correspondence

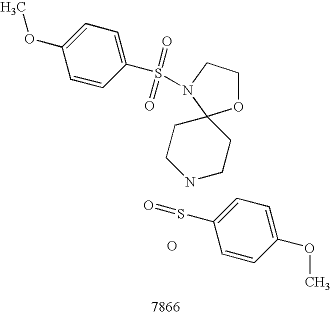

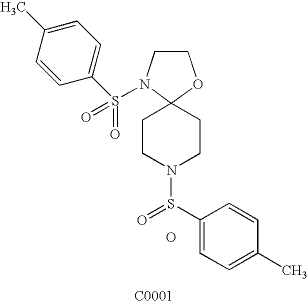

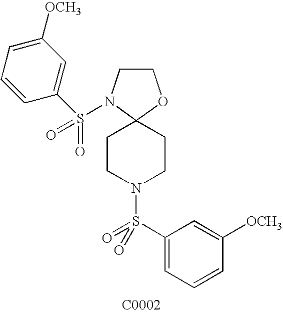

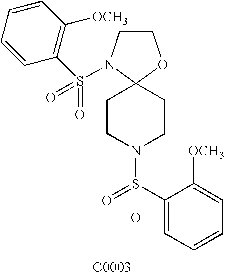

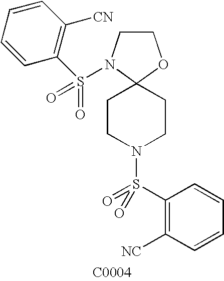

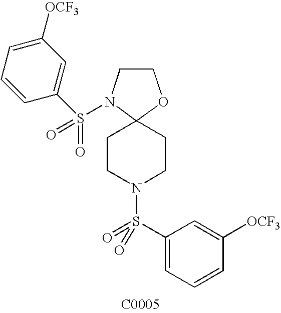

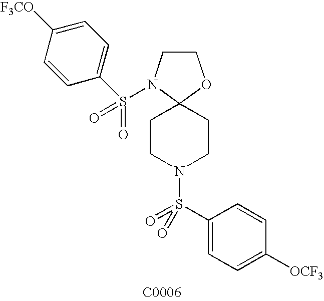

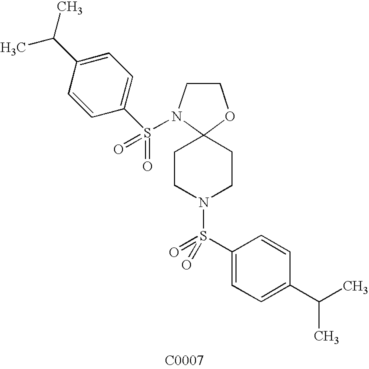

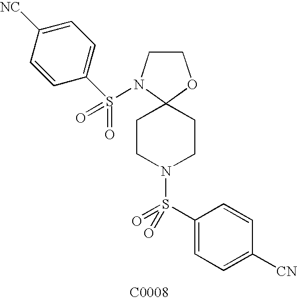

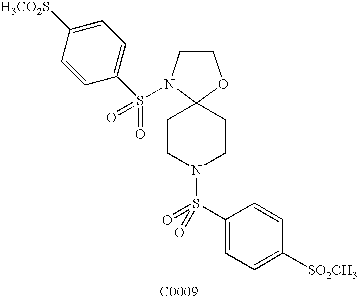

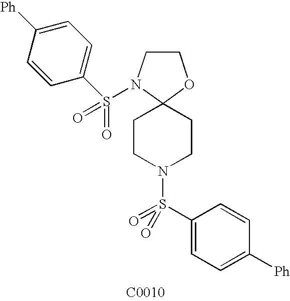

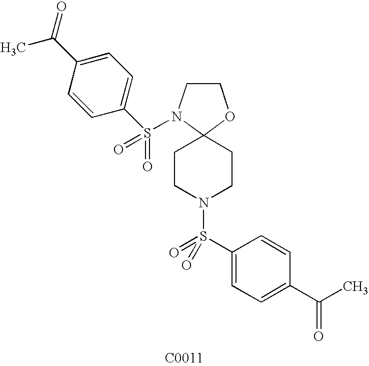

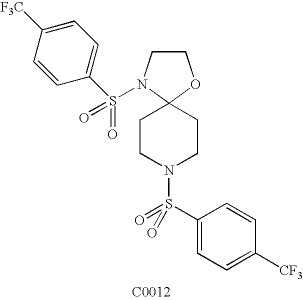

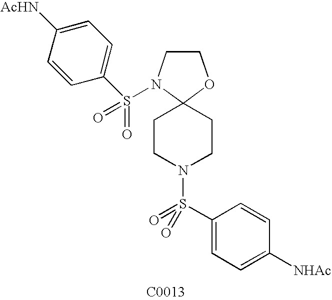

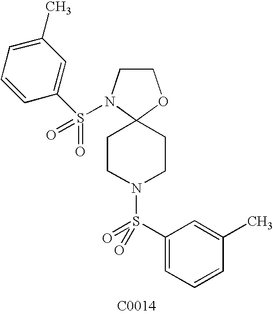

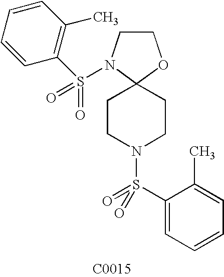

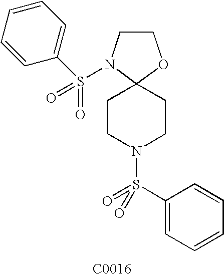

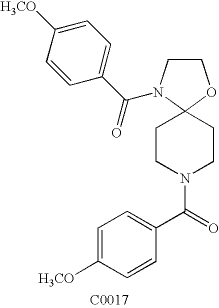

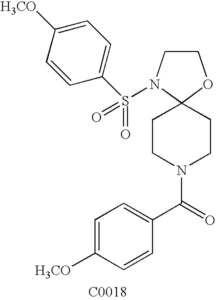

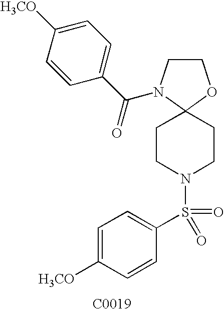

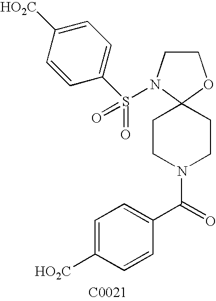

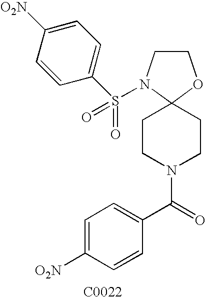

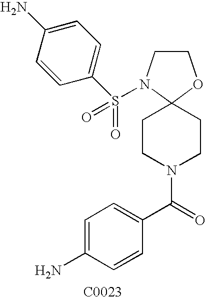

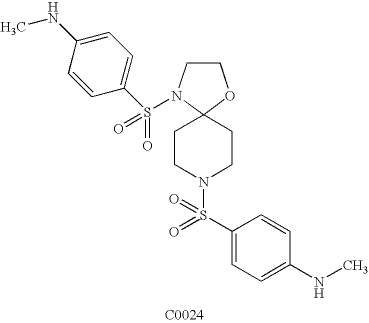

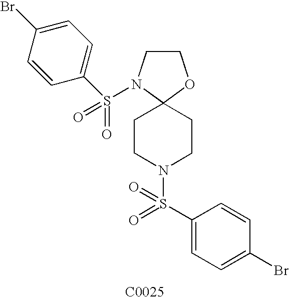

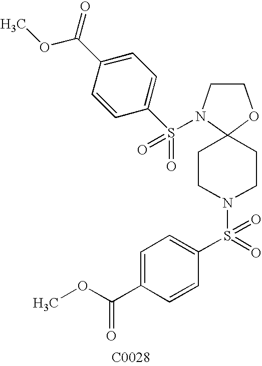

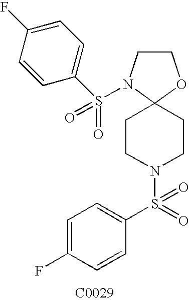

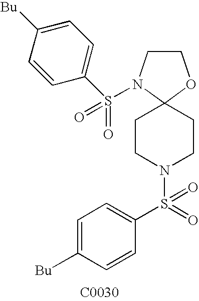

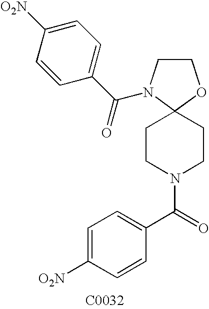

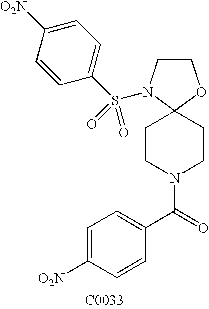

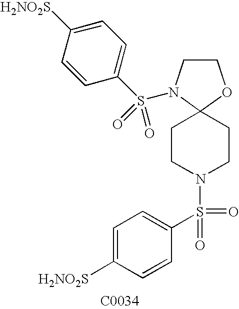

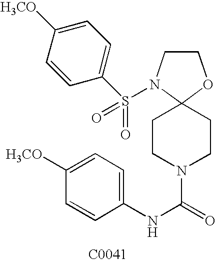

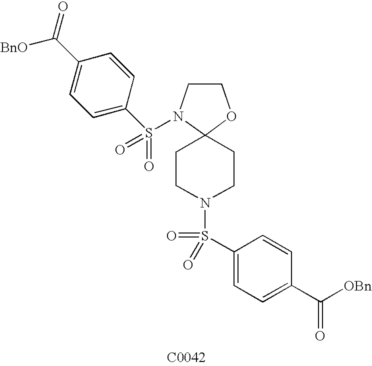

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

### Example 1

### MOR Agonist Activity

Using GTPγS Binding Assay

To assess the mu opiate receptor (MOR) agonist activity of positive compounds from the FLNA screening, compounds were tested in a [³⁵S]GTPγS binding assay using striatal membranes. Our previous study has shown that in striatal membranes, activation of MOR leads to an increase in [³⁵S]GTPγS binding to Gαo (Wang et al., 2005 Neuroscience 135:247-261).

Striatal tissue was homogenized in 10 volumes of ice cold 25 mM HEPES buffer, pH 7.4, which contained 1 mM EGTA, 100 mM sucrose, 50 μg/ml leupeptin, 0.04 mM PMSF, 2 μg/ml soybean trypsin inhibitor and 0.2% 2-mercaptoethanol. The homogenates were centrifuged at 800×g for 5 minutes and the supernatants were centrifuged at 49,000×g for 20 minutes. The resulting pellets were suspended in 10 volume of reaction buffer, which contained 25 mM HEPES, pH 7.5, 100 mM NaCl, 50 μg/ml leupeptin, 2 μg/ml soybean trypsin inhibitor, 0.04 mM PMSF and 0.02% 2-mercaptomethanol.

The resultant striatal membrane preparation (200 μg) was admixed and maintained (incubated) at 30° C. for 5 minutes in reaction buffer as above that additionally contained 1 mM MgCl₂ and 0.5 nM [³⁵S]GTPγS (0.1 μCi/assay, PerkinElmer Life and Analytical Sciences) in a total volume of 250 μl and continued for 5 minutes in the absence or presence of 0.1-10 μM of an assayed compound of interest. The reaction was terminated by dilution with 750 μl of ice-cold reaction buffer that contained 20 mM MgCl₂ and 1 mM EGTA and immediate centrifugation at 16,000×g for 5 minutes.

The resulting pellet was solubilized by sonicating for 10 seconds in 0.5 ml of immunoprecipitation buffer containing 0.5% digitonin, 0.2% sodium cholate and 0.5% NP-40. Normal rabbit serum (1 μl) was added to 1 ml of lysate and incubated at 25° C. for 30 minutes. Nonspecific immune complexes were removed by incubation with 25 μl of protein A/G-conjugated agarose beads at 25° C. for 30 minutes followed by centrifugation at 5,000×g at 4° C. for 5 minutes. The supernatant was divided and separately incubated at 25° C. for 30 minutes with antibodies raised against Gao proteins (1:1,000 dilutions).

The immunocomplexes so formed were collected by incubation at 25° C. for 30 minutes with 40 μl of agarose-conjugated protein A/G beads and centrifugation at 5,000×g at 4° C. for 5 minutes. The pellet was washed and suspended in buffer containing 50 mM Tris-HCl, pH 8.0, and 1% NP-40. The radioactivity in the suspension was determined by liquid scintillation spectrometry. The specificity of MOR activation of [³⁵S]GTPγS binding to Gao induced by a selective compound was defined by inclusion of 1 μM β-funaltrexamine (β-FNA; an alkylating derivative of naltrexone that is a selective MOR antagonist). DAMGO (H-Tyr-D-Ala-Gly-N-MePhe-Gly-OH; 1 or 10 μM) was used as a positive control.

The results of this study are shown in the Table below.

FLNA-Binding Compound MOR Agonist Activity

| FLNA- | Concentration of FLNA-Binding Compound as Agonist | | | | | |
|---|---|---|---|---|---|---|
| Binding | | | 1 μM + | % DAMGO | % DAMGO | % DAMGO + |
| Compound | 0.1 μM | 1 μM | BFNA | (0.1 μM) | (1 μM) | BFNA |
| 7866 | 152.3% | 308.2% | 62.4% | 79.3% | 94.8% | 129.5% |
| C0001 | 129.3% | 184.3% | 33.9% | 75.2% | 66.6% | 52.9% |
| C0002 | 88.4% | 93.8% | 3.9% | 51.4% | 33.9% | 6.1% |
| C0003 | 162.3% | 215.9% | 107.7% | 91.9% | 83.3% | 163.9% |
| C0004 | 122.0% | 228.4% | 65.8% | 72.1% | 85.4% | 99.7% |
| C0005 | 180.4% | 227.2% | 166.4% | 105.4% | 85.1% | 319.4% |
| C0006 | 121.5% | 204.0% | 4.6% | 70.6% | 73.8% | 7.2% |
| C0007 | 79.1% | 195.0% | 10.9% | 46.0% | 70.5% | 17.0% |
| C0008 | 71.2% | 201.6% | 2.8% | 41.4% | 72.9% | 4.4% |
| C0009 | 146.3% | 256.2% | 26.4% | 85.1% | 92.6% | 41.2% |
| C0010 | 136.5% | 307.0% | 89.1% | 80.7% | 114.9% | 135.0% |
| C0011 | 217.0% | 305.0% | 19.0% | 126.8% | 114.3% | 36.5% |
| C0012 | 96.8% | 224.8% | 184.4% | 54.8% | 86.7% | 280.7% |
| C0013 | 156.6% | 301.2% | 39.6% | 91.0% | 108.9% | 61.8% |
| C0014 | 144.9% | 153.5% | 76.3% | 82.0% | 59.2% | 116.1% |
| C0015 | 138.7% | 204.7% | 126.8% | 78.5% | 78.9% | 193.0% |
| C0016 | 172.7% | 230.5% | 96.7% | 100.4% | 83.3% | 150.9% |
| C0017 | 153.8% | 284.5% | 94.1% | 87.1% | 109.7% | 143.2% |
| C0018 | 195.5% | 247.7% | 106.5% | 110.7% | 95.5% | 162.1% |
| C0019 | 104.4% | 176.6% | 52.8% | 59.1% | 68.1% | 80.4% |
| C0021 | 159.7% | 192.0% | 90.7% | 94.5% | 87.8% | 546.4% |
| C0022 | 194.3% | 328.7% | 13.4% | 113.5% | 123.2% | 25.7% |
| C0023 | 153.2% | 233.7% | 23.2% | 89.5% | 87.6% | 44.5% |
| C0024 | 178.4% | 229.6% | 59.3% | 92.8% | 84.1% | 135.1% |
| C0025 | 235.7% | 320.7% | 80.2% | 122.6% | 117.5% | 182.7% |
| C0028 | 93.9% | 132.4% | 78.4% | 55.6% | 60.5% | 472.3% |
| C0029 | 175.4% | 308.8% | 16.6% | 91.2% | 113.1% | 37.8% |
| C0030 | 150.3% | 226.8% | 95.0% | 96.0% | 98.0% | 291.4% |
| C0032 | 145.4% | 202.0% | 80.9% | 92.8% | 87.3% | 248.2% |
| C0033 | 134.5% | 186.4% | 76.6% | 85.9% | 80.6% | 235.0% |
| C0034 | 103.6% | 167.9% | 80.1% | 61.3% | 76.7% | 482.5% |
| C0041 | 186.1% | 244.4% | 95.5% | 110.1% | 111.7% | 575.3% |
| C0042 | 167.1% | 260.9% | 110.6% | 98.9% | 119.2% | 666.3% |
| DAMGO | 168.5% | 266.1% | 53.2% | — | — | — |
| Average | | | | | | |

### Example 2

### FITC-NLX-Based FLNA Screening Assay

### A. Streptavidin-Coated 96-Well Plates

Streptavidin-coated 96-well plates (Reacti-Bind™ NeutrAvidin™ High binding capacity coated 96-well plate, Pierce-ENDOGEN) are washed three times with 200 μl of 50 mM Tris HCl, pH 7.4 according to the manufacturer's recommendation.

### B. N-Biotinylated VAKGL Pentapeptide VAKGL) (SEQ ID NO: 1)

Bn-VAKGL peptide (0.5 mg/plate) is dissolved in 50 μl DMSO and then added to 4450 μl of 50 mM Tris HCl, pH 7.4, containing 100 mM NaCl and protease inhibitors (binding medium) as well as 500 μl superblock in PBS (Pierce-ENDOGEN) [final concentration for DMSO: 1%].

### C. Coupling of Bn-VAKGL Peptides to Streptavidin-Coated Plate

The washed streptavidin-coated plates are contacted with 5 μg/well of Bn-VAKGL (100 μl) for 1 hour (incubated) with constant shaking at 25° C. [50 μl of Bn-VAKGL peptide solution from B+50 μl binding medium, final concentration for DMSO: 0.5%]. At the end of the incubation, the plate is washed three times with 200 μl of ice-cold 50 mM Tris HCl, pH 7.4.

### D. Binding of FITC-Tagged Naloxone [FITC-NLX] to VAKGL

Bn-VAKGL coated streptavidin plates are incubated with 10 nM fluorescein isothiocyanate-labeled naloxone (FITC-NLX; Invitrogen) in binding medium (50 mM Tris HCl, pH 7.4 containing 100 mM NaCl and protease inhibitors) for 30 minutes at 30° C. with constant shaking. The final assay volume is 100 μl. At the end of incubation, the plate is washed twice with 100 μl of ice-cold 50 mM Tris, pH 7.4. The signal, bound-FITC-NLX is detected using a DTX-880 multi-mode plate reader (Beckman).

### E. Screening of Medicinal Chemistry Analogs

The compounds are first individually dissolved in 25% DMSO containing 50 mM Tris HCl, pH 7.4, to a final concentration of 1 mM (assisted by sonication when necessary) and then plated into 96-well compound plates. To screen the medicinal chemistry analogs (new compounds), each compound solution (1 μl) is added to the Bn-VAKGL coated streptavidin plate with 50 μl/well of binding medium followed immediately with addition of 50 μl of FITC-NLX (total assay volume/well is 100 μl). The final screening concentration for each compound is 10 μM.

Each screening plate includes vehicle control (total binding) as well as naloxone (NLX) and/or naltrexone (NTX) as positive controls. Compounds are tested in triplicate or quadruplicate. Percent inhibition of FITC-NLX binding for each compound is calculated [(Total FITC-NLX bound in vehicle-FITC-NLX bound in compound)/Total FITC-NLX bound in vehicle]×100%]. To assess the efficacies and potencies of the selected compounds, compounds that achieve approximately 60-70% inhibition at 10 μM are screened further at 1 and 0.1 μM concentrations.

The results of this screening assay are shown in the table below.

FLNA Peptide Binding Assay

| FLNA-binding | Concentration of FLNA-binding Compound | | |
|---|---|---|---|
| Compound | 0.01 μM | 0.1 μM | 1 μM |
| 7866 | 38.5% | 47.9% | 53.4% |
| C0001 | 34.8% | 42.9% | 51.3% |
| C0002 | 38.4% | 45.6% | 42.8% |
| C0003 | 38.3% | 45.3% | 48.8% |
| C0004 | 37.6% | 42.3% | 44.7% |
| C0005 | 35.2% | 44.5% | 51.5% |
| C0006 | 41.6% | 46.8% | 51.8% |
| C0007 | 40.5% | 46.3% | 48.9% |
| C0008 | 42.2% | 52.3% | 54.4% |
| C0009 | 41.7% | 49.0% | 53.9% |
| C0010 | 39.8% | 42.7% | 47.1% |
| C0011 | 37.6% | 41.4% | 46.0% |
| C0012 | 26.3% | 39.5% | 46.4% |
| C0013 | 39.6% | 42.4% | 49.1% |
| C0014 | 29.5% | 38.8% | 40.0% |
| C0015 | 31.2% | 40.6% | 45.5% |
| C0016 | 38.3% | 43.8% | 49.1% |
| C0017 | 28.9% | 35.4% | 40.7% |
| C0018 | 42.3% | 45.9% | 53.4% |
| C0019 | 30.1% | 38.2% | 43.6% |
| C0021 | 34.0% | 38.4% | 40.6% |
| C0022 | 34.5% | 37.6% | 43.9% |
| C0023 | 35.9% | 41.7% | 47.2% |
| C0024 | 37.9% | 46.4% | 50.4% |
| C0025 | 37.2% | 41.4% | 45.1% |
| C0028 | 32.2% | 36.6% | 43.3% |
| C0029 | 38.6% | 43.2% | 50.5% |
| C0030 | 37.4% | 45.4% | 56.0% |
| C0032 | 41.5% | 50.5% | 55.3% |
| C0033 | 43.9% | 48.4% | 51.3% |
| C0034 | 29.6% | 38.3% | 44.8% |
| C0041 | 38.3% | 47.0% | 51.2% |
| C0042 | 42.4% | 49.7% | 56.1% |
| Naloxone Average | 40.61% | 47.75% | 51.54% |

### Example 3

### Tail-Flick Test

The mouse “tail flick” test was used to assay the relative antinociceptive activity of compositions containing a compound to be assayed. This assay was substantially that disclosed by Xie et al., 2005 J. Neurosci 25:409-416.

The mouse hot-water tail-flick test was performed by placing the distal third of the tail in a water bath maintained at 52° C. The latency until tail withdrawal from the bath was determined and compared among the treatments. A 10 second cutoff was used to avoid tissue damage. Data are converted to percentage of antinociception by the following formula: (response latency−baseline latency)/(cutoff−baseline latency)×100 to generate dose-response curves. Linear regression analysis of the log dose-response curves was used to calculate the A₅₀ (dose that resulted in a 50% antinociceptive effect) doses and the 95% confidence intervals (CIs). Relative potency was determined as a ratio of the A₅₀ values. The significance of the relative potency and the confidence intervals are determined by applying the t test at p<0.05.

To assess tolerance to the antinociceptive effect, the compound was administered twice daily for 7 days at an A₉₀ dose (dose that results in a 90% antinociceptive effect in the 52° C. warm-water tail-flick test), and the tail-flick test was performed daily after the a.m. dose. A significant reduction in tail-flick latency on subsequent days compared to the Day 1 administration of the A₉₀ dose indicates antinociceptive tolerance.

Orally administered morphine exhibited an A₅₀ value of 61.8 (52.4-72.9) mg/kg, and a mean maximum antinoniception amount of about 43% at 56 mg/kg at about 20 minutes. Orally administered compound C0011 exhibited a mean maximum antinoniception amount of about 70% at 56 mg/kg at about 10-20 minutes, whereas orally administered compound C0009 exhibited a mean maximum antinoniception amount of about 50% at 56 mg/kg at about 10 minutes, compound C0047 exhibited a mean maximum antinoniception amount of about 35% at 56 mg/kg at about 20-30 minutes, compound C0052 a mean maximum antinoniception amount of about 30% at 56 mg/kg at about 20 minutes, and compound C0022 exhibited a mean maximum antinoniception amount of about 40% at 56 mg/kg at about 30 minutes

### Example 4

### Dependence Test

On day 8, 16-20 hours after the last administration of an assay composition, animals were given naloxone to precipitate withdrawal (10 mg/kg, s.c.) before being placed in an observation chamber for 1 hour. A scale adapted from MacRae et al., 1997 Psychobiology 25:77-82 was used to quantify four categories of withdrawal behaviors: “wet dog” shakes, paw tremors, mouth movements, and ear wipes. Scores are summed to yield a total withdrawal score across the 1-hour test.

### Example 5

### Relative Gs/Go Switching

In this set of studies, the rat brain slice organotypic culture methods were modified from those published previously (Adamchik et al., 2000 Brain Res Protoc 5:153-158; Stoppini et al., 1991 J Neurosci Methods 37:173-182). Striatal slices (200 μM thickness) were prepared using a Mcllwain tissue chopper (Mickle Laboratory Engineering Co., Surrey, UK). Slices were carefully transferred to sterile, porous culture inserts (0.4 μm, Miilicell-CM) using the rear end of a glass Pasteur pipette. Each culture insert unit contained 2 slices and was placed into one well of the 12-well culture tray. Each well contain 1.5 ml of culture medium composed of 50% MEM with Earl's salts, 2 mM L--glutamine, 25% Earl's balanced salt solution, 6.5 g/l D-glucose, 20% fetal bovine serum, 5% horse serum, 25 mM HEPES buffer, 50 mg/ml streptomycin and 50 mg/ml penicillin. The pH value was adjusted to 7.2 with HEPES buffer.

Cultures were first incubated for 2 days to minimize the impact of injury from slice preparation. Incubator settings throughout the experiment were 36° C. with 5% CO₂. To induce tolerance, culture medium was removed and the culture insert containing the slices was gently rinsed twice with warm (37° C.) phosphate-buffered saline (pH 7.2) before incubation in 0.1% fetal bovine serum-containing culture medium with 100 μM morphine for 1 hour twice daily (at 9-10 AM and 3-4 PM) for 7 days.

Slices were returned to culture medium with normal serum after each drug exposure. Tissues were harvested 16 hours after the last drug exposure by centrifugation.

For determination of MOR-G protein coupling, slices were homogenated to generate synaptic membranes. Synaptic membranes (400 μg) were incubated with either 10 μM oxycodone or Kreb's-Ringer solution for 10 minutes before solubilization in 250 μl of immunoprecipitation buffer (25 mM HEPES, pH 7.5; 200 mM NaCl, 1 mM EDTA, 50 μg/ml leupeptin, 10 μg/ml aprotinin, 2 μg/ml soybean trypsin inhibitor, 0.04 mM PMSF and mixture of protein phosphatase inhibitors). Following centrifugation, striatal membrane lysates were immunoprecipitated with immobilized anti-Gαs/olf or -Gαo conjugated with immobilized protein G-agarose beads. The level of MOR in anti-Gαs/olf or -Gαo immunoprecipitates was determined by Western blotting using specific anti-MOR antibodies.

To measure the magnitude of MOR-mediated inhibition of cAMP production, brain slices were incubated with Kreb's-Ringer (basal), 1 μM DAMGO, 1 μM forskolin or 1 μM DAMGO+1 μM forskolin for 10 minutes at 37° C. in the presence of 100 μM of the phosphodiesterase inhibitor IBMX. Tissues were homogenized by sonication and protein precipitated with 1M TCA. The supernatant obtained after centrifugation was neutralized using 50 mM Tris, pH 9.0. The level of cAMP in the brain lysate was measured by a cAMP assay kit (PerkinElmer Life Science, Boston) according to manufacturer's instructions.

| | | | Gs/Go-Coupled |
|---|---|---|---|
| Condition | Gs/olf | Go | Ratio |
| Vehicle | | | |
| Average | 330.7 | 1996.4 | 0.173 |
| SEM | 34.6 | 192.0 | 0.34 |
| Oxycodone, | | | |
| 10 μM | | | |
| Average | 1425.2 | 900.4 | 1.588 |
| SEM | 77.8 | 26.2 | 0.103 |
| C0011, | | | |
| 10 μM | | | |
| Average | 534.3 | 1603.3 | 0.332 |
| SEM | 51.8 | 68.5 | 0.023 |
| C0011, | | | |
| 100 μM | | | |
| Average | 658.2 | 1598.8 | 0.420 |
| SEM | 34.2 | 114.9 | 0.030 |

A compound useful herein can be readily synthesized. An illustrative synthetic scheme is shown below that preparation of compounds containing two sulfonyl linkages and one sulfonyl and one carbonyl linkage. That scheme can be readily adapted for the preparation of compounds containing two carbonyl linkages and one carbonyl and one sulfonyl linkage in the opposite configurations from those shown. More detailed syntheses are set out hereinafter.

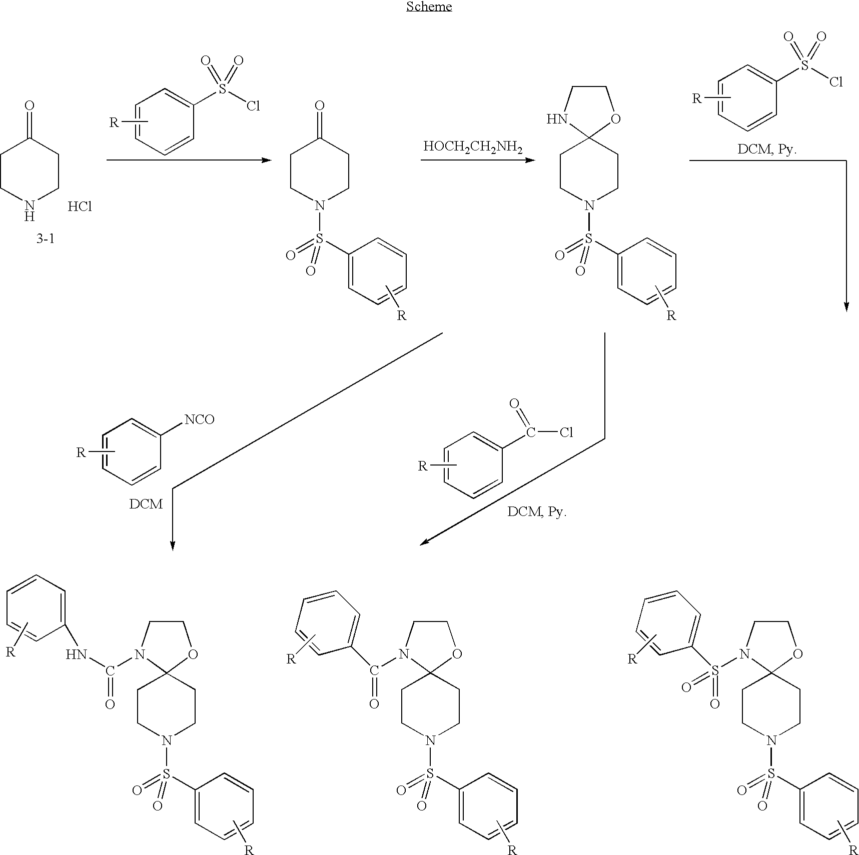

### Preparation of Compound C0001

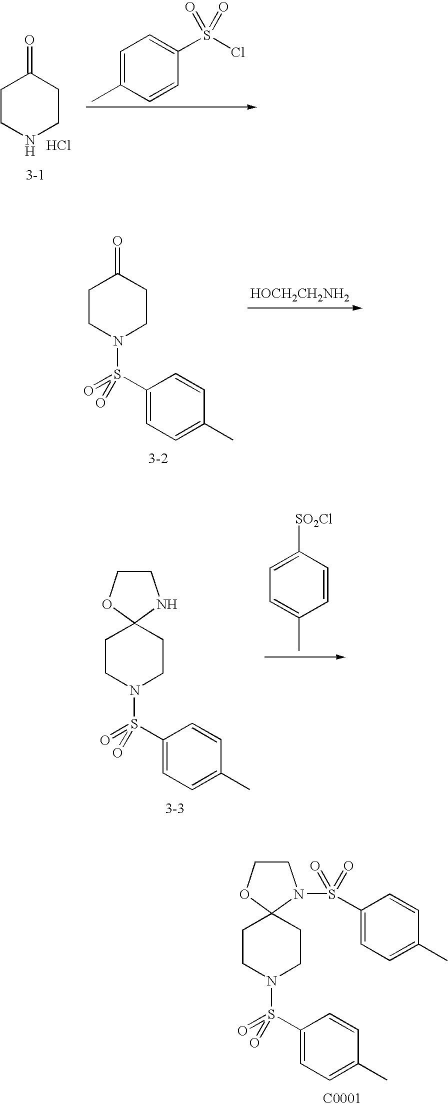

### Compound 3-2

To a solution of compound 3-1 (0.8 g, 5.23 mmol) in pyridine (20 mL) was added 4-methylbenzene-1-sulfonyl chloride (1.04 g, 5.49 mmol) in an atmosphere of N₂ and the mixture was allowed to react overnight (about 18 hours) at room temperature. Water was added and the resulting reaction mixture was extracted with CH₂Cl₂ 3 times. The combined organic layers were washed with 3M HCl and brine and concentrated to give compound 3-2 (0.78 g, yield: 59%, NMR confirmed).

### Compound 3-3

A solution of compound 3-2 (250 mg, 0.99 mmol), TsOH (20 mg) and 2-aminoethanol (5 mL) in EtOH (20 mL) was stirred overnight (about 18 hours) at room temperature. The solvent was removed under reduced pressure and the residue was partitioned between ethyl acetate and water. The organic layer was washed with water and brine, dried with Na₂SO₄ and concentrated to give compound 3-3 (230 mg, yield: 80%, NMR confirmed) as a white solid.

### Compound C0001

To a solution of compound 3-3 (180 mg, 0.61 mmol) in pyridine (15 mL) was added 4-methylbenzene-1-sulfonyl chloride (139 mg, 0.73 mmol) in an atmosphere of N₂ and the mixture was allowed to react at room temperature for 4 hours. Water was added and the resulting reaction mixture was extracted with CH₂Cl₂ 3 times. The combined organic layers were washed with 3M HCl and brine and concentrated to give the crude product (180 mg) as a red solid. Further purification gave compound C0001 (150 mg, yield: 54%, NMR confirmed, HPLC 94.5%) as a yellow solid.

### Preparation of C0002

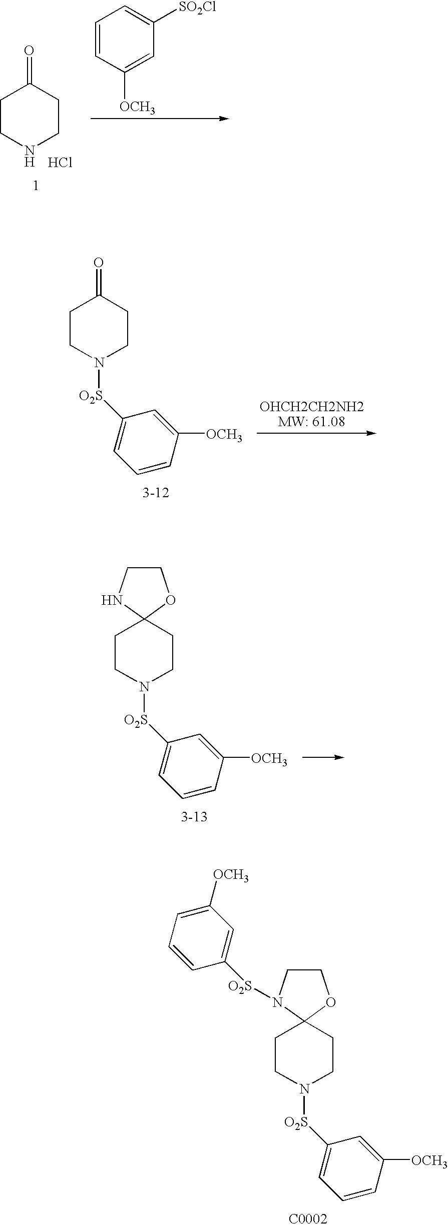

### Compound 3-12

A solution of compound 1 (300 mg, 2.21 mmol) in pyridine (8 mL) was admixed 4-methoxy-sulfonylbenzene-1-sulfonyl chloride (0.34 mL, 2.21 mmol). The mixture was stirred at room temperature for 3 hours. To the solution was added water and then extracted with DCM for 3 times. The combined organic phase was washed with 3M HCl and concentrated to give 335 mg of white solid (H NMR confirmed, 56% yield).

### Compound 3-12

A solution of compound 3-12 (335 mg, 1.244 mmol) in EtOH (10 mL) was treated with TsOH (25 mg) and HOCH₂CH₂NH₂ (2 mL). The mixture was stirred at room temperature overnight (about 18 hours). Then EtOH was removed under reduced pressure. The residue was partitioned between DCM and water. The organic phase was washed by saturated NaHCO₃ and brine then concentrated to provide 380 mg of colorless oil (yield 97.7%).

### Compound C0002

A solution of compound 3-13 (380 mg, 1.216 mmol) in Pyridine (8 mL) was treated with 4-methoxy-sulfonylbenzene-1-sulfonyl chloride (0.17 mL, 1.216 mmol). The mixture was stirred at room temperature overnight (about 18 hours). To the solution was added water and then extraction with DCM 3 times. The combined organic phase was washed with 3M HCl and concentrated to give 548 mg of crude product that was then purified to give 450 mg of light yellow powder (MS and H NMR confirmed, HPLC 95.3%, yield 76.7%). ¹H-NMR (400MHz, CDC;₃) δ: 7.46-7.41 (m, 3H), 7.35-7.32 (m, 2H), 7.27-7.25 (m, 1H), 7.13-7.10 (m, 2H), 3.89-3.86 (m, 8H), 3.78-3.76 (m, 2H), 3.51 (t, J=6.4 Hz, 2H), 2.60-2.51 (m, 4H), 1.65-1.60 (m, 2H); MS (ESI) calcd for C₂₁H₂₆N₂O₇S₂ (m/z): 482.12. found: 483.3 [M+1]⁺, 505.3 [M+23]⁺.

### Preparation of Compound C0003

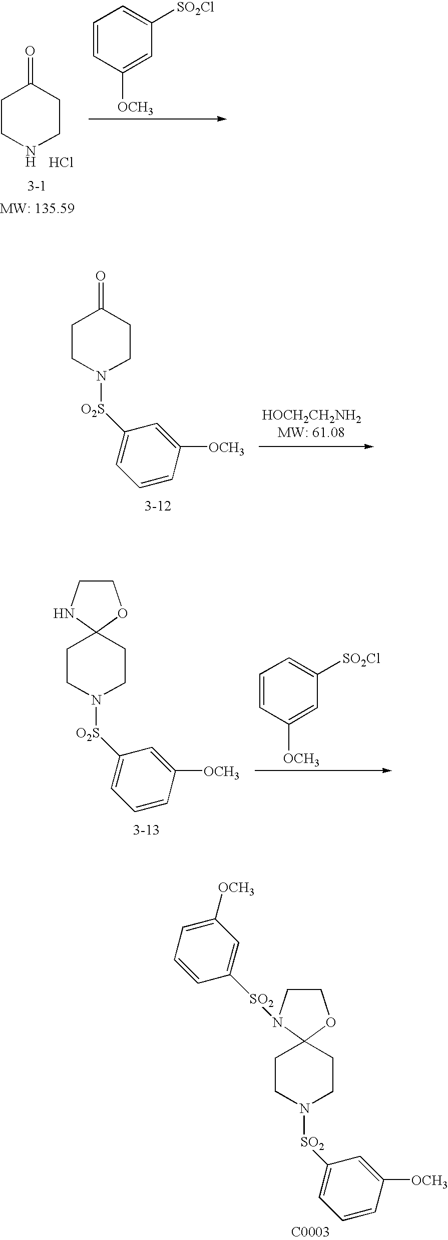

### Compound 3-12

To a solution of compound 3-1 (300 mg, 2.21 mmol) in pyridine (8 mL) was added 4-methoxysulfonyl-benzene-1-sulfonyl chloride (0.34 mL, 2.21 mmol) and the reaction mixture was stirred at room temperature for 3 hours. Water was added and the resulting reaction mixture was extracted with CH₂Cl₂ 3 times. The combined organic layers were washed with 3M HCl and concentrated to give compound 3-12 (335 mg, yield: 56%, NMR confirmed) as a white solid.

### Compound 3-13

To a solution of compound 3-12 (335 mg, 1.244 mmol) in EtOH (10 mL) was added TsOH (25 mg) and 2-aminoethanol (2 mL) and the reaction mixture was stirred overnight (about 18 hours) at room temperature. EtOH was removed under reduced pressure and the residue was partitioned between CH₂Cl₂ and water. The organic phase was washed with saturated NaHCO₃ and brine and concentrated to give compound 3-13 (380 mg, yield: 97.7%) as a colorless oil.

### Compound C0003

To a solution of compound 3-13 (380 mg, 1.216 mmol) in pyridine (8 mL) was added 4-methoxy-sulfonylbenzene-1-sulfonyl chloride (0.17 mL, 1.216 mmol) and the reaction mixture was stirred overnight (about 18 hours) at room temperature. Water was added and the resulting reaction mixture was extracted with CH₂Cl₂ 3 times. The combined organic layers were washed with 3M HCl and concentrated to give the crude product (548 mg) which was further purified to give compound C0003 (450 mg, yield: 76.7%, MS and NMR confirmed, HPLC 95.3%) as a light yellow powder

### Preparation of Compound C0004

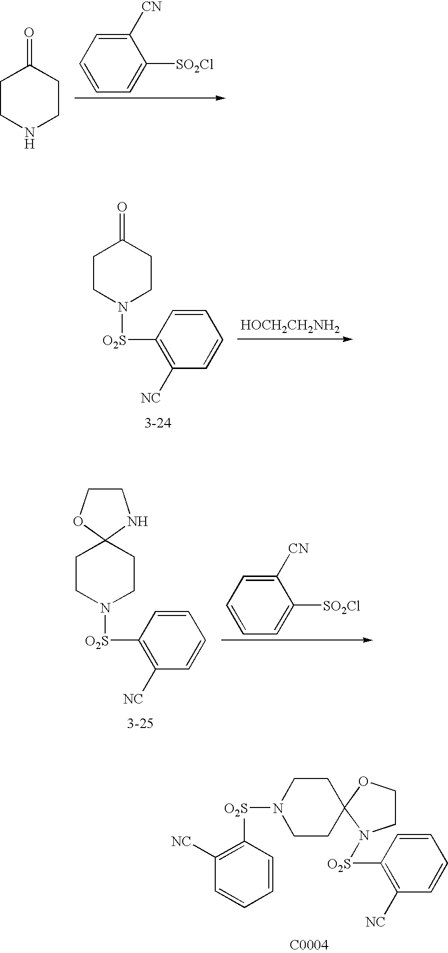

### Preparation of Compound 3-25

To a solution of compound 3-24 (35 mg, 0.13 mmol) in ethanol (10 ml) was added 2-aminoethanol (0.5 ml) and p-toluene sulfonyl acid monohydrate (5 mg). The mixture was stirred at 30° C. overnight (about 18 hours). The solvent was then removed by evaporation under vacuum. To the residue was added CH₂Cl₂ (30 ml), then the CH₂Cl₂ layer was washed with saturated Na₂CO₃ (15 mL×2) and water (20 mL×3), dried over Na₂SO₄ and concentrated to give the crude product as yellow oil (33 mg, yield: 80.5%, ¹H-NMR confirmed).

### Compound C0004

To a solution of compound 3-25 (33 mg, 0.11 mmol) in pyridine (5 ml) was added o-cyanobenzene sulfonyl chloride (26 mg, 0.13 mmol). The mixture was stirred overnight (about 18 hours) at room temperature. Then the solvent was removed under reduced pressure. The residue was diluted with CH₂Cl₂ (20 ml), washed with 3M HCl (10 ml×3), and the organic layer was dried, and the solvent evaporated to give the crude product as yellow oil. The crude product was purified with silica gel column to give the title product as light yellow solid (8 mg, yield 15.8%, HPLC 95.2%, ¹H-NMR and MS confirmed).

### Preparation of Compound C0005

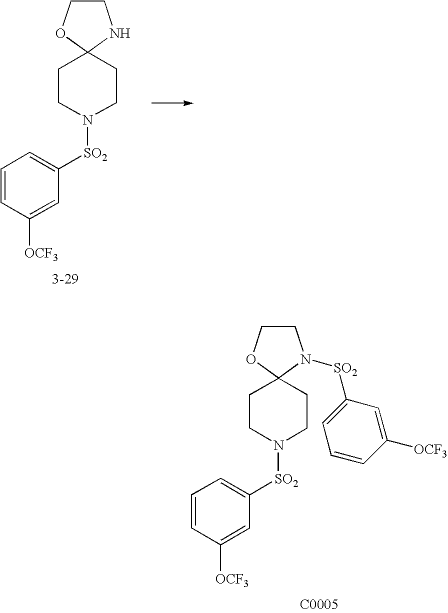

To a solution of compound 3-29 (145 mg, 0.4 mmol) in pyridine (2 mL) was added 3-trifluoro-methoxybenzenesulfonyl chloride (103 mg, 1.1 mmol). The mixture was then stirred at room temperature overnight (about 18 hours). Water was added then the mixture was extracted with DCM 3 times. The combined organic phase was washed with 3M HCl and concentrated to get the crude product. The crude product was purified to afford 40 mg of the desired product as white solid (1H NMR and LC-MS confirmed, HPLC 94.4%, yield).

### Preparation of Compound C0006

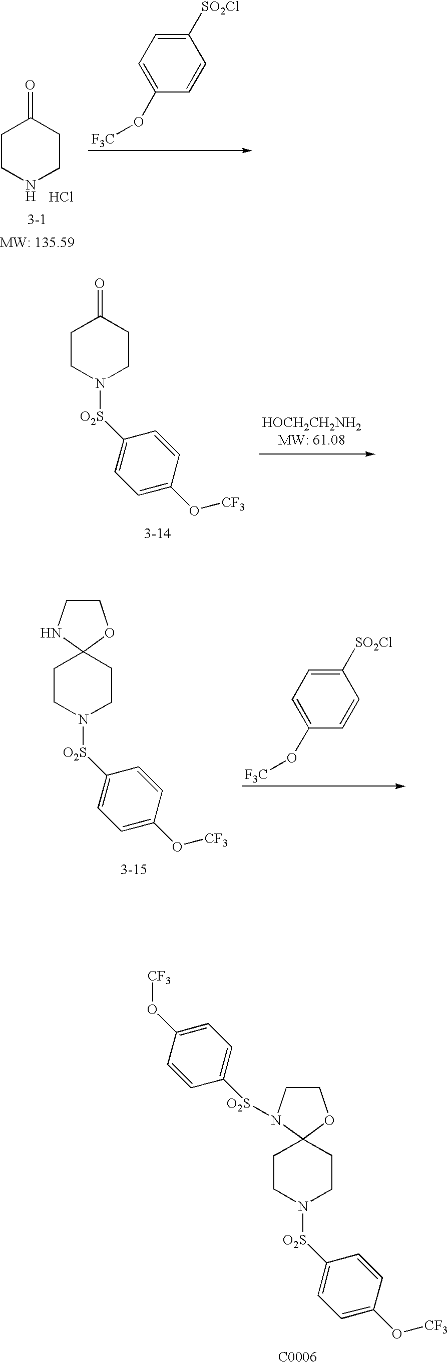

### Compound 3-14

To a solution of compound 3-1 (100 mg, 0.7375 mmol) in pyridine (3 mL) was added 4-trifluoromethoxybenzene-1-sulfonyl chloride (192.38 mg, 0.7375 mmol) and the reaction mixture was stirred at room temperature for 3 hours. Water was added and the resulting reaction mixture was extracted with CH₂Cl₂ 3 times. The combined organic layers were washed with 3M HCl and concentrated to give compound 3-14 (111 mg, yield: 46.6%, NMR confirmed) as a white solid.

### Compound 3-15

To a solution of compound 3-14 (111 mg, 0.343 mmol) in EtOH (4 mL) was added TsOH (10 mg) and 2-aminoethanol (1 mL) and the reaction mixture was stirred at room temperature for 4 hours. EtOH was removed under reduced pressure and the residue was partitioned between CH₂Cl₂ and water. The organic layer was washed with saturated aqueous NaHCO₃ and brine and concentrated to give compound 3-15 (128 mg of crude compound, yield: >100%, NMR confirmed) as a light yellow liquid.

### Compound C0006

To a solution of compound 3-15 (128 mg, 0.349 mmol) in pyridine (2.5 mL) was added 4-trifluoromethoxybenzene-1-sulfonyl chloride (91 mg, 0.349 mmol) and the reaction mixture was stirred at room temperature for 3 hours. Water was added and the resulting reaction mixture was extracted with CH₂Cl₂ 3 times. The combined organic layers were washed with 3M HCl and concentrated to give the crude product (132 mg) which was further purified by column chromatography over silica gel to afford compound C0006 (95 mg, yield: 46%, NMR and MS confirmed, HPLC 99%).

### Preparation of Compound C0007

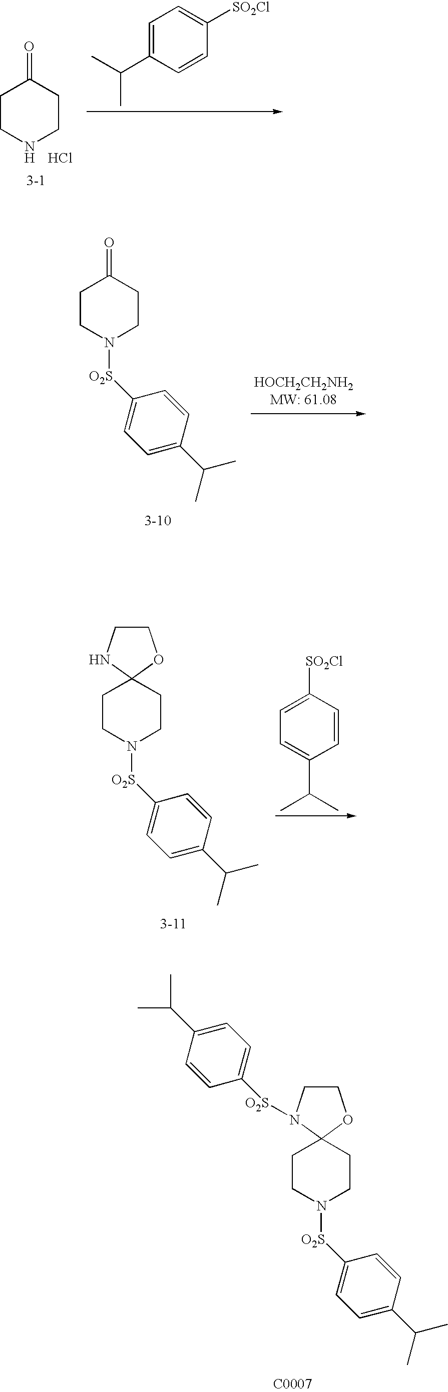

### Compound 3-10

To a solution of compound 3-1 (100 mg, 0.7375 mmol) in pyridine (3 mL) was added 4-isopropylsulfonylbenzene-1-sulfonyl chloride (0.13 mL, 0.7375 mmol) and the reaction mixture was stirred at room temperature for 3 hours. Water was added and the resulting reaction mixture was extracted with CH₂Cl₂ 3 times. The combined organic layers were washed with 3M HCl and concentrated to give compound 3-10 (105 mg, yield: 50.7%, NMR confirmed) as a white solid.

### Compound 3-11

To a solution of compound 3-10 (200 mg, 0.71 mmol) in EtOH (6 mL) was added TsOH (15 mg) and 2-aminoethanol (1.5 mL) and the reaction mixture was stirred overnight (about 18 hours) at room temperature. EtOH was removed under reduced pressure and the residue was partitioned between CH₂Cl₂ and water. The organic phase was washed with saturated aqueous NaHCO₃ and brine and concentrated to give compound 3-11 (231 mg, yield: 100%) as a white foam.

### Compound C0007

To a solution of compound 3-11 (300 mg, 0.925 mmol) in pyridine (8 mL) was added 4-isopropylsulfonylbenzene-1-sulfonyl chloride (0.17 mL, 0.925 mmol) and the reaction mixture was stirred overnight (about 18 hours) at room temperature. Water was added and the resulting reaction mixture was extracted with CH₂Cl₂ 3 times. The combined organic layers were washed with 3M HCl and concentrated to give the crude product (384 mg) as a yellow oil (MS confirmed, HPLC 84%, yield: 82.1). The crude product was triturated in ether/hexane system and filtered to give compound C0007 (240 mg, yield: 51.3%, MS and NMR confirmed, HPLC 95.0%) as a light yellow powder.

### Preparation of Compound C0008

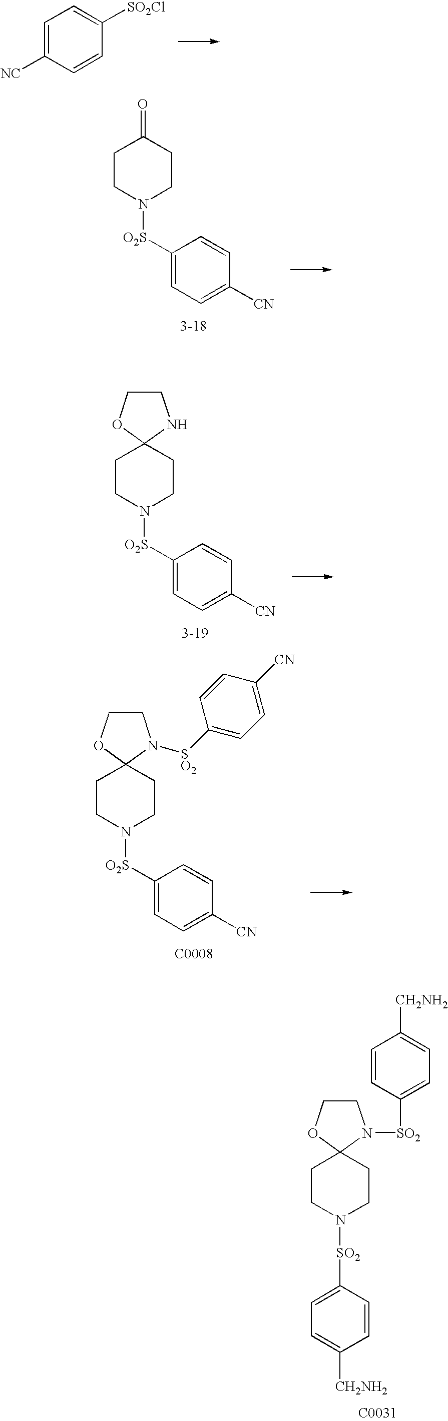

### Compound 3-18

To a solution of piperidin-4-one (354 mg, 2.31 mmol) in pyridine (10 ml) was added 4-cyanobenzene-1-sulfonyl chloride (310 mg, 1.54 mmol). The mixture was stirred overnight (about 18 hours) at room temperature. The solvent was then removed under reduced pressure. The residue was diluted with CH₂Cl₂ (100 ml), washed with 2N HCl (50 mL×3), dried over anhydrous Na₂SO₄ and concentrated to give the crude product as a yellow solid (138 mg, yield: 34%, TLC confirmed).

### Compound 3-19

To a solution of compound 3-18 (138 mg, 0.52 mmol) in ethanol (20 ml) was added 2-aminoethanol (2 mL) and p-toluenesulfonyl acid monohydrate (20 mg). The mixture was stirred at 20° C. overnight (about 18 hours). The solvent was then removed under reduced pressure. The residue was diluted with CH₂Cl₂ (60 mL), washed with saturated Na₂CO₃ (50 mL×3), dried over anhydrous Na₂SO₄ and concentrated to give the title compound as a yellow solid (0.15 g, yield:94%, TLC confirmed).

### Compound C0008

To a solution of compound 3-19 (150 mg, 0.49 mmol) in pyridine (10 ml) was added 4-cyanobenzene-1-sulfonyl chloride (147 mg, 0.73 mmol). The mixture was stirred at room temperature overnight (about 18 hours). The solvent was removed under reduced pressure. The residue was diluted with CH₂Cl₂ (50 mL), washed with 2N HCl (30 ml×3), dried over anhydrous Na₂SO₄ and concentrated to give the crude product as a yellow solid. The crude product was purified with a silica gel column to give the pure product as a light yellow solid (100 mg, yield: 43%, TLC confirmed).

### Preparation of Compound C0009

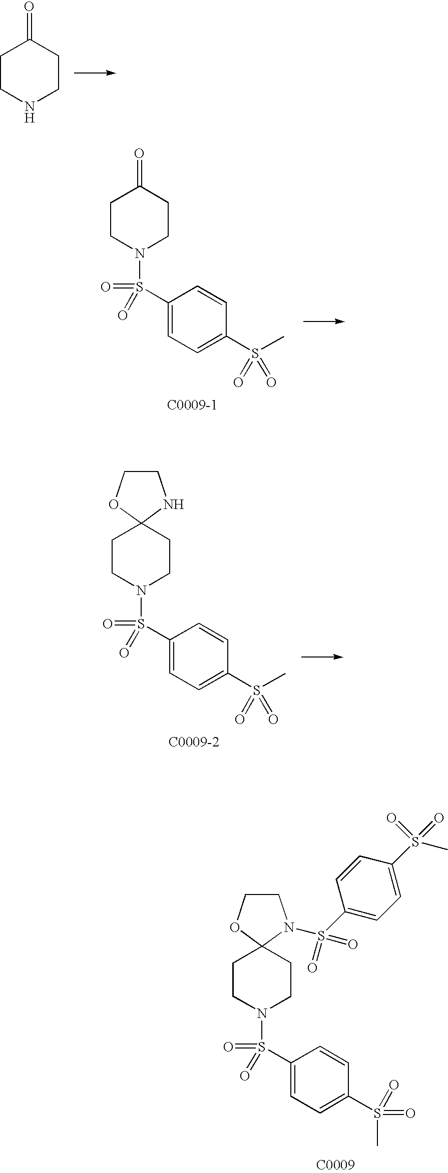

### Compound C0009

To a solution of compound C0009-2 (570 mg, 1.58 mmol) in pyridine (20 mL) was added 4-methyl-sulfonyl-benzene sulfonyl chloride (604 mg, 2.37 mmol). The mixture was then stirred overnight (about 18 hours) at room temperature. The solvent was then removed under reduced pressure. The crude product was then diluted with CH₂Cl₂ (250 mL) and washed with 1M HCl (100mL×2), and the aqueous layer was extracted with CH₂Cl₂ (100 mL). The organic phase was then dried over anhydrous Na₂SO₄ and concentrated and then the crude product was recrystallized from DCN to give 150 mg purified product as a light yellow solid (¹H-NMR and MS confirmed, HPLC: 96%). The solution was then evaporated to give 200 mg of the purified product. The pure product was then re-purified with silica gel column to give C0009 as a light yellow solid (180 mg, yield: 33%, ¹H-NMR and MS confirmed, HPLC: 96%).

### Preparation of Compound C0010

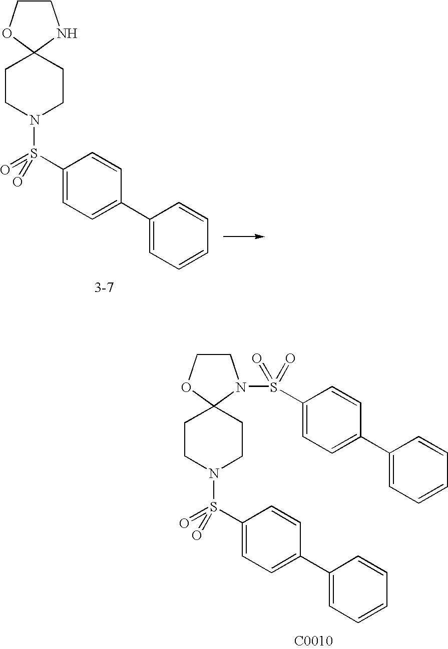

To a solution of compound 3-7 (202 mg, 0.564 mmol) in pyridine (4 mL) was added 4-phenyl-benzenesulfonyl chloride (142 mg, 0.564 mmol). The mixture was stirred at room temperature overnight (about 18 hours). To the solution was added water and then the solution was extracted with DCM 3 times. The combined organic phase was washed with 3M HCl then concentrated to give 234 mg of crude product. The crude product was purified by silica gel column to afford 68 mg of pure product (LC-MS and 1H NMR showed this is a mixture of compound 3-7 and desired product). Further purification by silica gel column eluted by (CH3OH:DCM=100:1) gave 55 mg of the desired product with 86% purity. This product was again purified by Pre-TLC to give the desired product with 90% purity.

### Preparation of Compound C0011

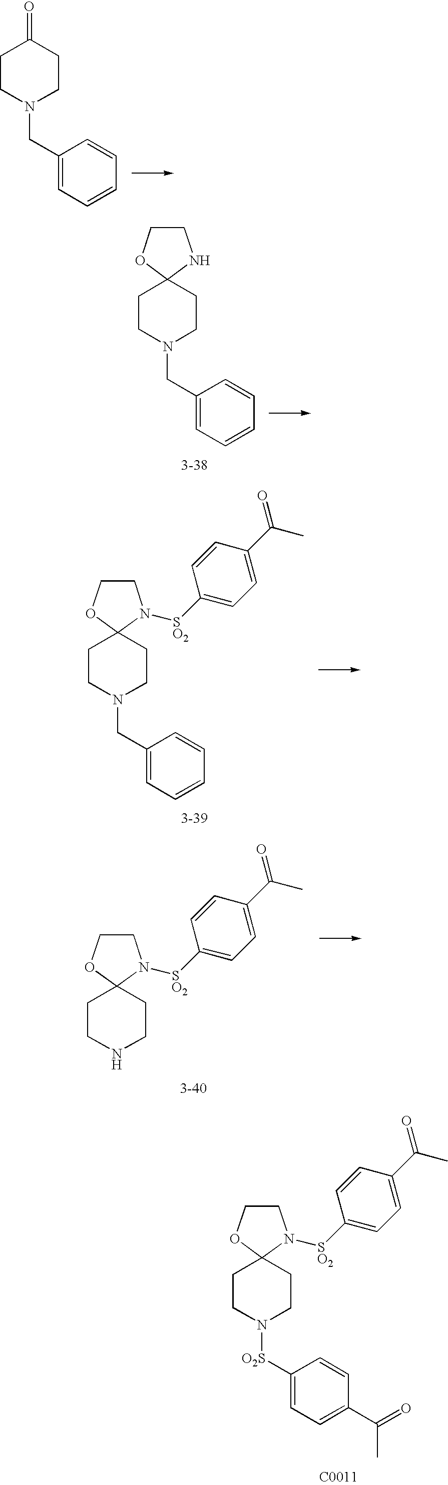

### Compound 3-38

To a solution of N-benzyl-4-piperidone (3.8 g, 20.1 mmol) in ethanol (30 mL) was added 2-aminoethanol (2.45 g, 40.2 mmol) and p-toluene sulfonyl acid monohydrate (0.1 g). The mixture was stirred at 30° C. overnight (about 18 hours). The solvent was removed under reduced pressure. To the residue was added CH₂Cl₂ (100 mL) and saturated Na₂CO₃ (60 mL). The CH₂Cl₂ layer was separated and washed with saturated Na₂CO₃ (50 mL×4). Then the organic layer was dried over Na₂SO₄ and concentrated to give the crude product as a brown oil (3 g, yield: 63.8%, ¹H-NMR confirmed).

### Compound 3-39

To a solution of compound 3-38 (382 mg, 1.65 mmol) in pyridine (10 mL) was added p-acetyl-benzenesulfonyl chloride (300 mg, 1.37 mmol). The mixture was stirred at room temperature overnight (about 18 hours). The solvent was removed under reduced pressure. To the residue was added CH₂Cl₂ (50 mL), then the solution was washed with saturated Na₂CO₃ aqueous (30 mL×3), dried over Na₂SO₄ and concentrated to give the crude product as brown oil.

### Compound 3-40

To a solution of compound 3-39 (1.33 g, 3.2 mmol) in MeOH/CH₂Cl₂ (40/20 ml) was added 10% Pd/C (270 mg). The mixture was stirred under H₂ at room temperature for 24 hours. TLC indicated that no reaction had taken place. Then the Pd/C was replaced with Pd(OH)₂/C, and the reaction was stirred under H₂ at room temperature and atmosphere pressure overnight (about 18 hours). TLC indicated that the reaction completed. The reaction mixture was filtrated and evaporated to give the crude product as light yellow solid (0.98 g, yield: 93.6%, LC-MS confirmed).

### Compound C0011

To the solution of compound 3-40 (700 mg, 2.16 mmol) in pyridine (20 ml) was added 4-acetylbenzene-1-sulfonyl chloride (566 mg, 2.59 mol). The mixture was stirred at room temperature for 2d. The solvent was removed under reduced pressure. The residue was diluted with 50 mL DCM and washed with 2N HCl (100mL×3). The organic layer was dried over anhydrous Na₂SO₄ and concentrated to give the crude product as yellow solid, which was purified with silica gel column to give the product as yellow solid (510 mg, yield: 46.6%, Lot#: MCO334-28-1, LC-MS confirmed).

### Preparation of Compound C0012

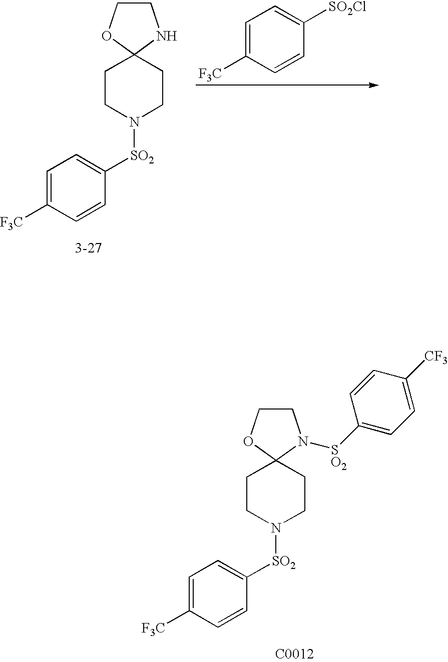

To a solution of compound 3-27 (144 mg, 0.41 mmol) in pyridine (2 mL) was added 4-trifluoromethylbenzene-1-sulfonyl chloride (101 mg, 0.41 mmol). The mixture was stirred at room temperature overnight (about 18 hours). Water was added to that solution then extracted with DCM 3 times. The combined organic phase was washed with 3M HCl and concentrated to get the crude product. The crude product was purified to give 40 mg of the desired product (1H NMR confirmed, HPLC 95%, 17.5% yield).

### Preparation of Compound C0013

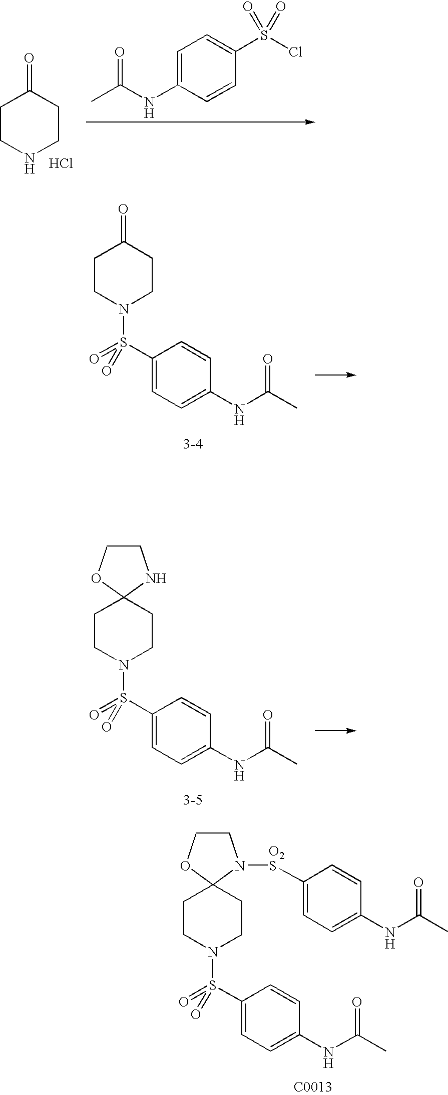

### Compound C0013

To a solution of compound 3-5 (0.59 g, 1.74 mmol) in pyridine (50 ml) was added 4-acetyl-aminobenzenesulfonyl chloride (0.49 g, 2.09 mmol). The mixture was stirred overnight (about 18 hours) at room temperature. The solvent was removed under reduced pressure. To the residue was added CH₂Cl₂ (100 mL) and 2N HCl (50 mL). The CH₂Cl₂ layer was separated and washed with 2N HCl (30 mL×2), then dried over anhydrous Na₂SO₄ and concentrated to give the crude product as yellow solid, which was purified with silica gel column to give the pure product as white solid (320 mg, yield:34.4%, HPLC: 97%).

### Preparation of Compound C0014

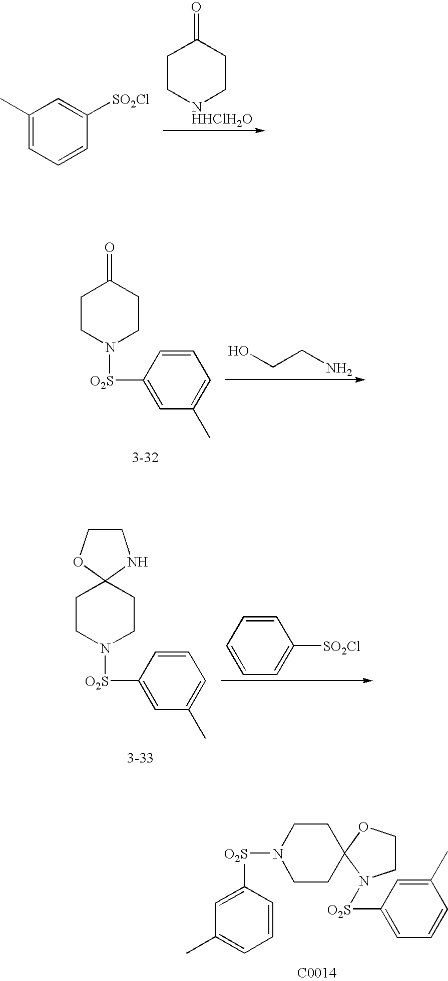

### Compound 3-33

A solution of compound 3-32 (140 mg, 0.55 mmol), p-toluene sulfonyl acid (15 mg) and 2-aminoethanol (2 ml) in ethanol (20 ml) was stirred overnight (about 18 hours) at room temperature. The solvent was removed by evaporation under vacuum. To the residue was added ethyl acetate (50 ml) and water (50 ml). The ethyl acetate layer was washed with water (30 ml×3), dried over Na₂SO₄ and concentrated to give the crude product as a yellow oil (170 mg, yield: 103.6%).

### Compound C0014

The compound m-methylbenzene sulfonyl chloride (131 mg, 0.69 mmol) was added to a solution of compound 3-33 (170 mg, 0.57 mmol) in pyridine (2 mL). The mixture was stirred overnight (about 18 hours) at room temperature. To the residue was added CH₂Cl₂ (50 mL). The organic solution was washed with 3M HCl (30 mL×3). Next, the CH₂Cl₂ layer was evaporated to give the title product as a yellow oil. The crude product was purified by silica gel column to give the pure product as a white powder (30 mg, yield: 11.63%, H-NMR and MS confirmed, HPLC 95.4%). About 50 mg of compound 3-32 was recovered as white powder.

### Preparation of Compound C0015

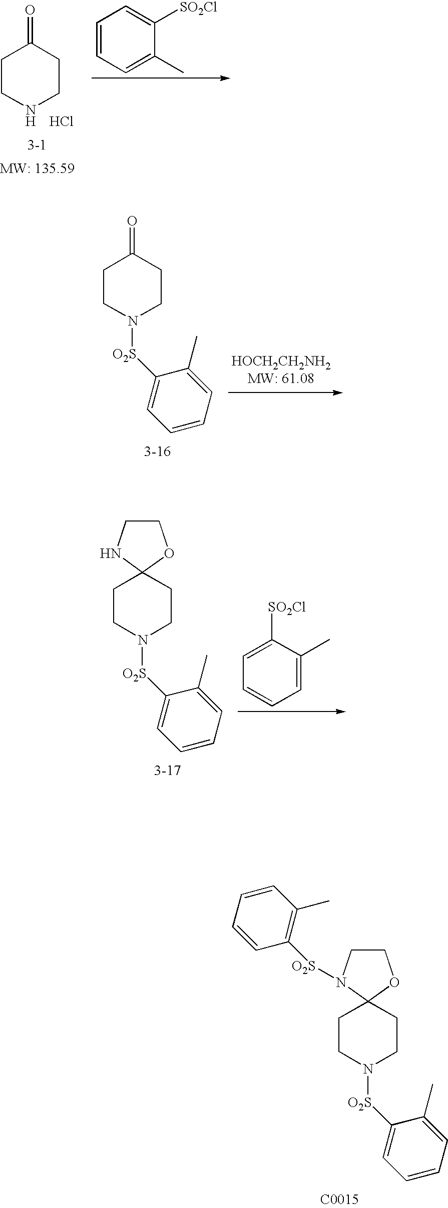

### Compound 3-16

To a solution of compound 3-1 (100 mg, 0.7375 mmol) in pyridine (3 mL) was added 2-methyl-benzene-1-sulfonyl chloride (140.6 mg, 0.7375 mmol) and the reaction mixture was stirred overnight (about 18 hours) at room temperature. Water was added and the resulting reaction mixture was extracted with CH₂Cl₂ 3 times. The combined organic layers were washed with 3M HCl and concentrated to give compound 3-16 (104 mg, yield: 56%, NMR confirmed) as a white solid.

### Compound 3-17

To a solution of compound 3-16 (104 mg, 0.41 mmol) in EtOH (4 mL) was added TsOH (10 mg) and 2-aminoethanol (1 mL) and the reaction mixture was stirred overnight (about 18 hours) at room temperature. EtOH was removed under reduced pressure and the residue was partitioned between CH₂Cl₂ and water. The organic phase was washed with saturated aqueous NaHCO₃ and brine and concentrated to give the crude compound 3-17 (120 mg, yield: 100%) as a light yellow liquid.

### Compound C0015

To a solution of compound 3-17 (100 mg, 0.405 mmol) in pyridine (2.5 mL) was added 2-methyl-benzene-1-sulfonyl chloride (77.2 mg, 0.405 mmol) and the reaction mixture was stirred overnight (about 18 hours) at room temperature. Water was added and the resulting reaction mixture was extracted with CH₂Cl₂ 3 times. The combined organic layers were washed with 3M HCl and concentrated to give the crude product (97 mg) that was further purified to provide compound C0015 (28 mg, yield: 15%, NMR and MS confirmed, HPLC 91%).

### Preparation of Compound C0016

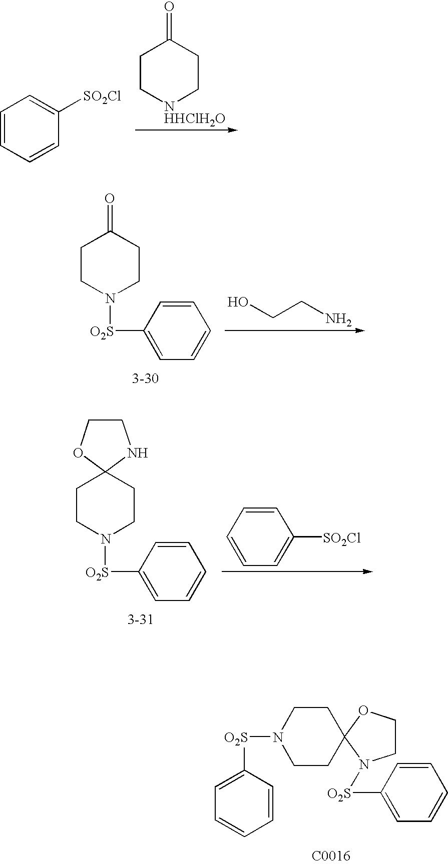

### Compound 3-30

To a solution of piperidin-4-one (208 mg, 1.36 mmol) in 20 mL of pyridine was added benzenesulfonyl chloride (200 mg, 1.13 mmol). The mixture was stirred at room temperature overnight (about 18 hours). The pyridine was then removed by evaporation under vacuum. To the residue was added CH₂Cl₂ (50 mL), then the CH₂Cl₂ layer was washed with 3M HCl (30 mL×3), dried over Na₂SO₄ and concentrated to give the crude product as a light yellow solid (138 mg, yield: 51%).

### Compound 3-31

A solution of compound 3-30 (136 mg, 0.57 mmol), p-toluene sulfonyl acid monohydrate (15 mg) and 2-aminoethanol (2 mL) in EtOH (20 mL) was stirred overnight (about 18 hours) at room temperature. The solvent was removed by evaporation under vacuum. To the residue was added ethyl acetate (50 mL) and water (50 mL). The ethyl acetate layer was washed with water (30 mL×3). The water phase was washed with ethyl acetate (20 mL). The combined organic layers were dried over Na₂SO₄, filtered and concentrated to give the crude product (151 mg, yield: 92.5%). The crude product was directly used in the next step.

### Compound C0016

To a solution of compound 3-31 (150 mg, 0.53 mmol) in pyridine (15 mL) was added phenyl sulfonyl chloride (112 mg, 0.64 mmol). The mixture was stirred at room temperature overnight (about 18 hours). The solvent was removed by evaporation under vacuum. To the residue was added CH₂Cl₂ (50 mL). The CH₂Cl₂ layer was washed with 3M HCl (30 mL×3), dried over Na₂SO₄ and concentrated to give the crude product as a light yellow solid. The crude product was purified with a silica gel column using petroleum ether/ethyl acetate 2:1 (PE/EA=2/1) solvent to give the pure product as white solid (97 mg, yield:43.3%, HPLC: 97% purity, ¹H-NMR and MS have confirmed).

### Preparation of Compounds C0017 and C0018

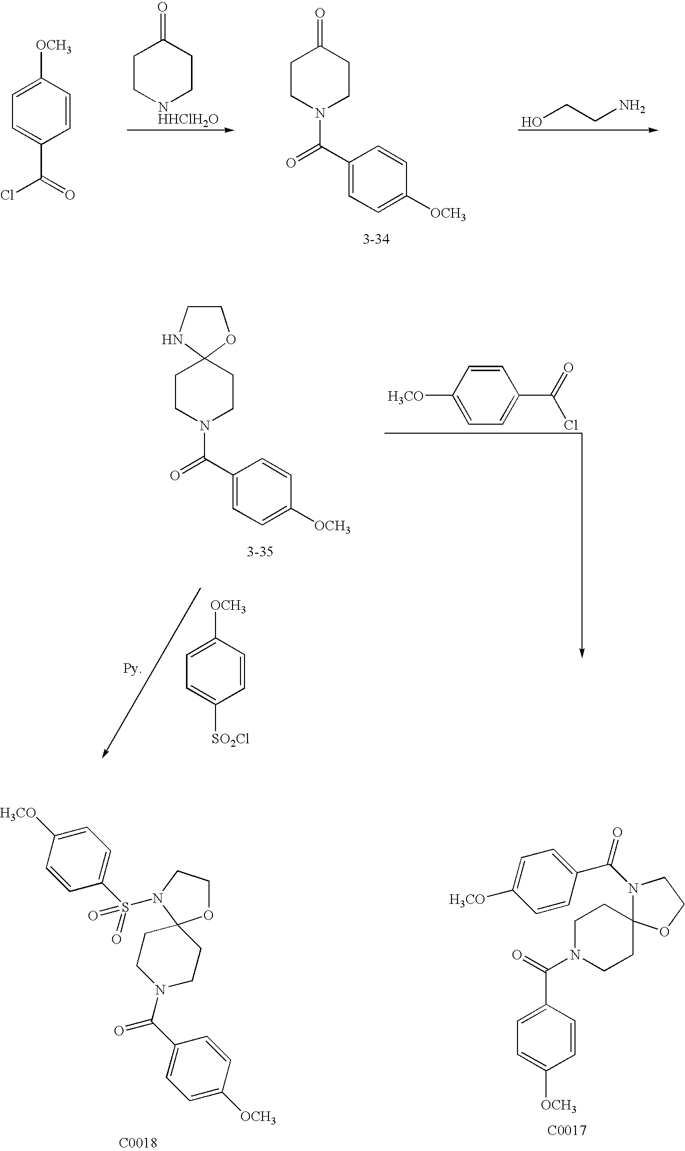

### Compound 3-34

To a solution of piperidin-4-one (0.37 g, 1.95 mmol) in pyridine (20 mL) was added 4-methoxybenzoyl chloride (0.5 g, 2.93 mmol). The reaction mixture was stirred at room temperature overnight (about 18 hours). The reaction solvent was then removed under reduced pressure. The residue was dissolved in CH₂Cl₂ (50 mL), then washed with 3M HCl (50 mL×3). The organic layer was dried over Na₂SO₄ and evaporated to give the title compound as a brown oil (330 mg, yield: 61.5%, LC-MS confirmed).

### Compound 3-35

A solution of compound 3-34 (330 mg, 1.42 mmol), 2-aminoethanol (2 ml) and p-toluenesulfonic acid monohydrate (33 mg) in ethanol (20 mL) was stirred at room temperature overnight (about 18 hours). The solvent was then removed by evaporation under reduced pressure. The residue was diluted with CH₂Cl₂ (50 mL), then washed with water (50 mL×3). The organic layer was dried over Na₂SO₄ and evaporated to give the crude product as a yellow oil (360 mg, yield: 92.1%, H-NMR and MS confirmed).

### Compound C0017

To a solution of compound 3-35 (172 mg, 0.62 mmol) in pyridine (25 mL) was added 4-methoxybenzoyl chloride (160 mg, 0.93 mmol). The reaction was stirred overnight (about 18 hours) at room temperature. The solvent was then removed under reduced pressure. The residue was diluted with CH₂Cl₂ (60 mL), then washed with 3M HCl (30 mL×3). The organic layer was dried over Na₂SO₄ and concentrated to give the crude product as a brown oil. The crude product was purified by silica gel column to give pure product as a white solid (220 mg, yield: 86%, H-NMR and MS confirmed, HPLC: 99.1%).

### Compound C0018

To a solution of compound 3-35 (198 mg, 0.72 mmol) in pyridine (25 mL) was added 4-methoxy-benzenesulfonyl chloride (220 mg, 1.07 mmol). The reaction mixture was stirred at room temperature overnight (about 18 hours). The reaction solvent was then removed under reduced pressure. The residue was diluted with CH₂Cl₂ (50 mL), then washed with 3M HCl (30 mL×3). The organic layer was dried over Na₂SO₄ and evaporated to give the crude product as a brown oil.

### Preparation of Compound C0019

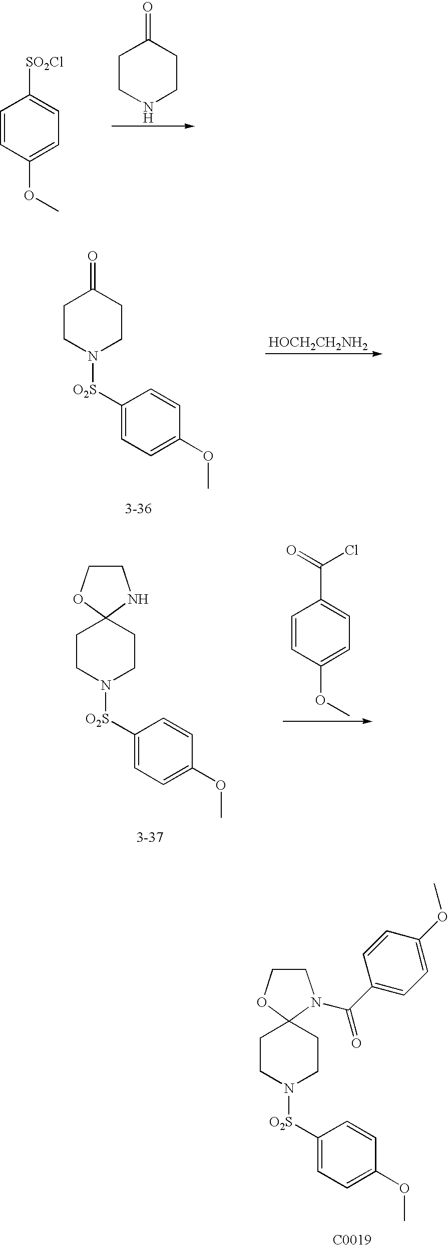

### Compound 3-36

To a solution of piperidine-4-one (178 mg, 1.16 mmol) in pyridine (20 mL) was added 4-methoxy-benzenesulfonyl chloride (200 mg, 0.97 mmol). The mixture was stirred at room temperature overnight (about 18 hours). The pyridine was then removed under reduced pressure. The residue was diluted with CH₂Cl₂ (50 mL), then washed with 3M HCl (30 mL×3). The organic layer was dried over anhydrous Na₂SO₄ and concentrated to give the product as a yellow solid (260 mg, yield: 100%, LC-MS confirmed).

### Compound 3-37

A solution of compound 3-36 (130 mg, 0.48 mmol), 2-aminoethanol (2 ml) and p-toluenesulfonic acid monohydrate (13 mg) in EtOH (20 mL) was stirred at room temperature overnight (about 18 hours). The solvent was removed under reduced pressure. The residue was dissolved in CH₂Cl₂ (50 mL), then washed with saturated Na₂CO₃ (50 mL×2) and water (50 mL×2). The organic layer was then dried over Na₂SO₄ and concentrated to give the product as a white colloid (118 mg, yield: 78.11, LC-MS confirmed)

### Compound C0019

To a solution of compound 3-37 (118 mg, 0.38 mmol) in pyridine (25 mL) was added p-methoxybenzoyl chloride (96.7 mg, 0.57mmol). The mixture was stirred overnight (about 18 hours) at room temperature. The pyridine was removed under reduced pressure. The residue was diluted with CH₂Cl₂ (50 ml), then washed with 3 M HCl (30 mL×3). The organic layer was dried over Na₂SO₄ and concentrated to give the crude product as a brown oil.

### Preparation of Compound C0021

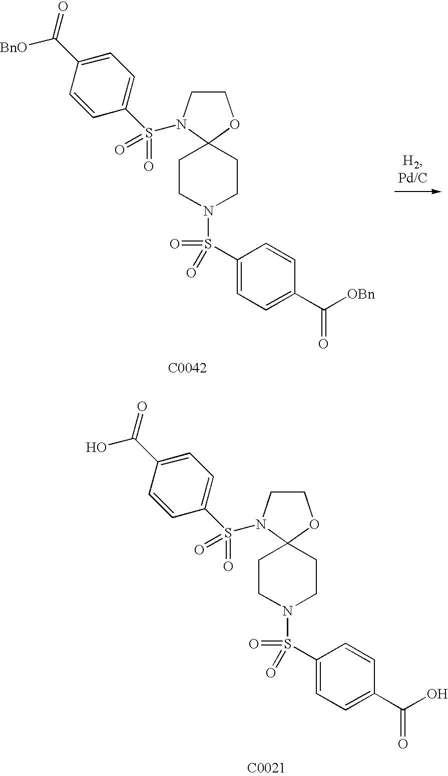

A solution of compound C0042 (110 mg, 0.16 mmol) in 10 mL methanol and 10 mL dichloromethane was added to 45 mg Pd/C, then the mixture was stirred at room temperature for 24 hours under H₂. TLC indicated the reaction was not complete, so the mixture was stirred at room temperature under H₂ (P=2.5M pa) for 2 more days. Later, TLC indicated that the starting material did not react. Next, Pd/C was replaced by Pd(OH)₂/C after hydrogenation under P=2.5 Mpa for 20 hours. Next, the mixture was filtered and the solvent was removed by reduced pressure evaporation to get the product as a white solid (60 mg, yield: 74%, confirmed by LC-MS, ¹HNMR and MASS, 97.8% purity by HPLC).

### Preparation of Compounds C0022 and C0023

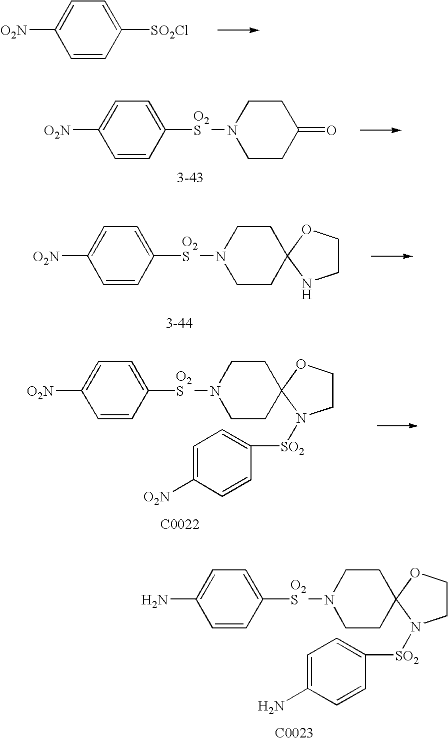

### Compound C0022

To a solution of compound 3-44 (220 mg, 0.67 mmol) in pyridine (15 mL), the compound 4-nitrobenzenesulfonyl chloride (218 mg, 0.99 mmol) was added and the reaction mixture was stirred at 30° C. for 72 hours. The solvent was then removed under reduced pressure and the residue was diluted with CH₂Cl₂ (30 mL). Next, the residue was washed with 3 N HCl (15 mL×3) and the organic layer was dried then evaporated to give the crude compound as a yellow solid. The crude material was purified with a silica gel column (E/P=1:2 to ethyl acetate) to get the pure product (210 mg, yield: 62.5%, HPLC: 97%, ¹H-NMR confirmed).

### Compound C0023

To a solution of C0022 (30 mg, 0.059 mmol) in MeOH (10 mL), 10% Pd/C was added (10 mg). The reaction mixture was stirred under H₂ overnight (about 18 hours). After the reaction was complete (checked by TLC), Pd/C was filtered off, and the filtrate was evaporated to get the crude compound (33 mg). The crude material was purified with a silica gel column (MC/MeOH=100:1) to obtain the desired compound as a white solid (23 mg, yield:88%, confirmed by ¹H-NMR). HPLC showed that the purity was 92%.

### Preparation of C0024

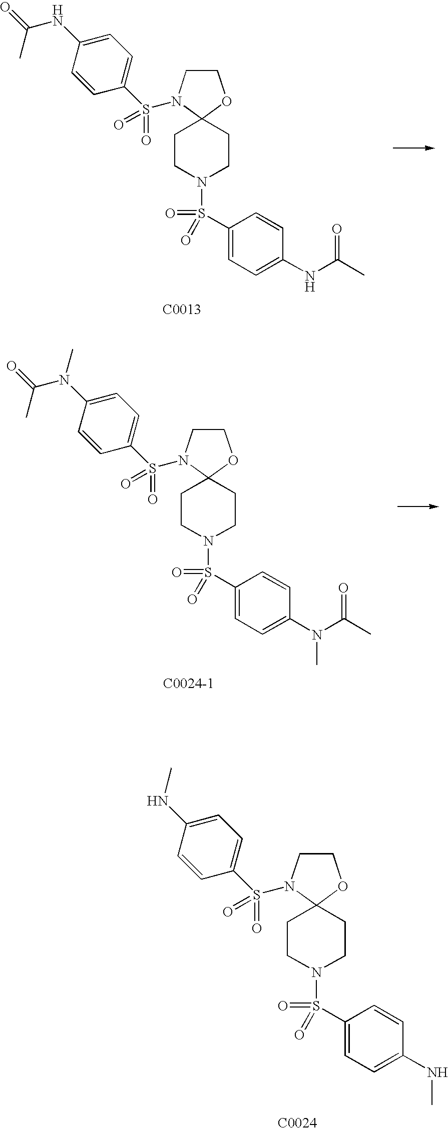

### Compound C0024-1

To a solution of compound C0013 (50 mg, 0.09 mmol) in THF (5 mL), 60% NaH (8.64 mg, 0.36 mmol) was added, the reaction mixture was stirred at room temperature for half an hour. Then CH₃I (0.16 mL, 0.54 mmol) was added. The mixture was stirred at room temperature overnight (about 18 hours). The reaction was quenched with MeOH. The solvent was removed under reduced pressure. The residue was diluted with water (20 mL), and extracted with CH₂Cl₂ (15 mL×3). The combined organic layer was dried over anhydrous Na₂SO₄, filtered, and evaporated to give the crude compound as yellow oil. Then the crude product was purified with silica gel column (eluted with EA/PE=1:1 to MeOH/DCM=1:100) to give two products: MC0287-19-1 (20 mg) and MC0287-19-2 (15 mg). After checking with ¹H-NMR and MASS, compound MC0287-19-1 was determined to be the de-diacetyl compound C0024, and MC0287-19-2 the de-monoacetyl compound C0024-2.

### Compound C0024

To a solution of compound MC0287-19-2 (C0024-2-1 or C0024-2-2, 15 mg) in MeOH (10 mL), NaOH was added. The reaction mixture was stirred at room temperature. After the starting material was gone (monitored by TLC), the solvent was removed to get the residue, that was diluted with CH₂Cl₂ (20 mL), washed with water (10 mL×3), the organic layer was dried over anhydrous Na₂SO₄, filtered, evaporated to give the crude compound. The crude product was purified by silica gel column (EA/PE=1:2 to 1:1) to get the pure compound (3 mg). It was combined with MC0287-19-1 and was purified to the give the pure product (20 mg, HPLC: 98. MS and ¹H-NMR confirmed). ¹H-NMR (400 MHz, CDCl₃) δ: 7.61 (d, J=10 Hz, 2H), 7.54 (d, J=9.2 Hz, 2H), 6.58 (t, J=9.6 Hz, 4H), 4.27-4.23 (m, 2H), 3.89-3.80 (m, 2H), 3.68 (d, J=8.8 Hz, 2H), 3.45 (t, J=6.4 Hz, 2H), 2.89 (bs, 6H), 2.53-2.43 (m, 4H), 1.60-1.57 (m, 2H); MS (ESI) calcd for C₂₁H₂₈N₄O₅S₂ (m/z): 480.15. found: 503.0 [M+23]⁺

### Preparation of Compounds C0025 and C0028

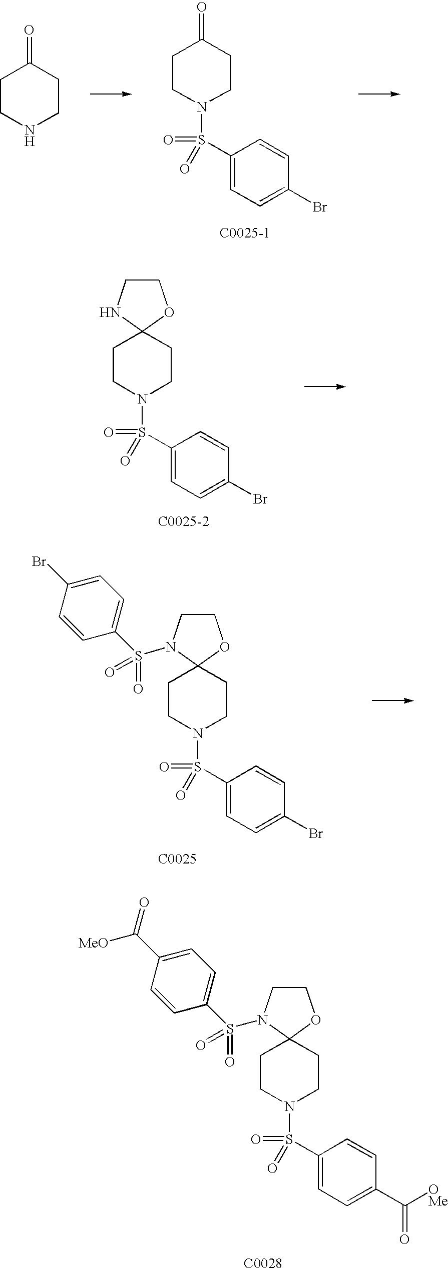

### Compound C0025-1

To a solution of piperidin-4-one (1.8 g, 11.74 mmol) in pyridine (30 ml) was added 4-bromobenzene-1-sulfonyl chloride (2 g, 7.83 mmol). The mixture was stirred overnight (about 18 hours) at room temperature. The solvent was removed under reduced pressure. The residue was diluted with CH₂Cl₂ (100 ml), washed with 3N HCl (100 ml×2), dried over anhydrous Na₂SO₄ and concentrated to give the title compound as a pale solid (1.3 g, yield: 521, TLC confirmed).

### Compound C0025-2

A solution of C0025-1 (1.3 g, 4.09 mmol), 2-aminoethanol (5 mL) and p-toluenesulfonic acid monohydrate (130 mg) was stirred overnight (about 18 hours) at 25° C. in 60 mL ethanol. The solvent was removed by reduced pressure evaporation. The residue was diluted with 200 mL dichloromethane, washed with water (100 mL×3) and saturated sodium bicarbonate solutions (100 mL×3). Next, the organic layer was dried and concentrated to get the product as a white solid. (1.44 g, yield: 97%, TLC confirmed).

### Compound C0025

To a solution of C0025-2 (1.44 g, 3.99 mmol) in 60 mL of pyridine, 4-bromobenzenesulfonyl chloride (1.53 g, 5.98 mmol) was added with stirring at room temperature overnight (about 18 hours). The solvent was removed under reduced pressure. The residue was diluted with 200 mL dichloromethane, and washed with 1 M hydrochloride (100 mL×3). The organic layer was then dried and concentrated to give the crude product as a yellow solid. The crude product was purified with a silica gel column and solvent of DCM:MeOH=500:1 to give the desired product as a yellow solid (0.3 g pure+0.7 g impure, yield: 43)

### Compound 28

To a solution of compound C0025 (100 mg, 0.17 mmol) in 20 mL DMF was added Pd(PPh₃)₄ (60 mg), triethylamine (0.1 mL) and methanol (8 mL), with stirring at 130° C. overnight (about 18 hours) under carbon monoxide (p=2M pa). The mixture was quenched with 5 mL water, and the solvent was removed under reduced pressure evaporation. The residue was diluted with 50 mL dichloromethane and washed with water (50 mL×3). The organic layer was dried and concentrated to obtain the crude product as a green solid. After purification with a silica gel column and solvent of DCM to DCM:MeOH=500:1, the purified product was obtained as a yellow solid (85 mg, yield: 91%, ¹H-NMR confirmed).

Repeat preparation: To a solution of compound C0025 (235 mg, 0.41 mmol) in 20 mL of DMF was added Pd(PPh₃)₄ (468 mg, 0.41 mmol), triethylamine (0.17 mL, 1.22 mmol) and 8 mL of methanol. Next, the mixture was stirred at 140° C. for 36 hours under pressure (P=2.5 Mpa). The reaction was quenched by the addition of 5 mL of water. The solvent was then removed under reduced pressure evaporation and the residue was diluted with 50 mL dichloromethane. The crude product was washed with water (50 mL×3). The organic layer was dried and concentrated to get the crude product as a green solid. After purified with a silica gel column (dichloromethane), the product was obtained as a yellow solid (65 mg, yield: 30%, TLC confirmed, HPLC: 84%).

### Preparation of Compound C0029

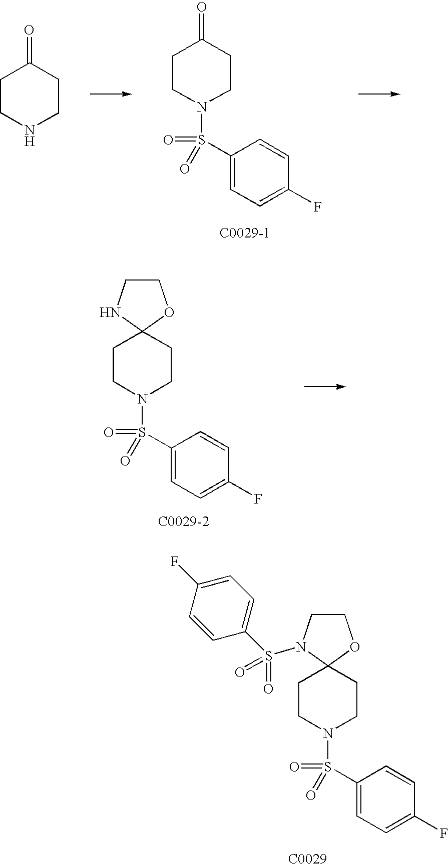

### Compound C0029-1

To a solution of piperidine-4-one (1.47 g, 7.71 mmol) in pyridine (20 mL), 4-flurobenzene-sulfonyl chloride (1 g, 5.14 mmol) was added, the reaction mixture was stirred at room temperature overnight (about 18 hours), the solvent was removed under the reduced pressure, the residue was diluted with CH₂Cl₂ (20 mL), washed with 3N HCl (15 mL×3), the organic layer was dried over anhydrous Na₂SO₄, filtered, evaporated to give the crude compound as white solid (0.72 g, yield: 54.5%, ¹H-NMR confirmed). ¹H-NMR (400 MHz, CDCl₃) δ: 7.82˜7.78 (m, 2H), 7.24˜7.20 (m, 2H), 3.38 (t, J=6 Hz, 4H), 3.54 (d, J=6 Hz, 4H).

### Compound C0029-2

A solution of compound C0029-1 (0.72 g, 2.8 mmol), 2-aminoethanol (0.26 g, 4.2 mmol) and p-toluenesulfonic acid monohydrous (100 mg) in ethanol (20 mL) was stirred at 25° C. overnight (about 18 hours). The solvent was removed under the reduced pressure. The residue was diluted with CH₂Cl₂ (20 mL), washed with NaHCO₃ solution (20 mL×3), the organic layer was dried over anhydrous Na₂SO₄, filtered, evaporated to give the crude compound as white solid (0.81 g, yield: 96%, ¹H-NMR confirmed). ¹H-NMR (400 MHz, CDCl₃) δ: 7.81-7.75 (m, 2H), 7.20-7.14(m, 2H), 3.67 (t, J=6.4 Hz, 2H), 3.31-3.26 (m, 2H), 3.12(t, J=6.4 Hz, 2H), 2.97-2.94 (m, 2H), 1.76-1.74 (m, 4H).

### Compound C0029

To a solution of compound C0029-2 (0.81 g, 2.7 mmol) in pyridine (20 mL), 4-flurobenzene-sulfonyl chloride (0.79 g, 4.06 mmol) was added. The reaction mixture was stirred at room temperature overnight (about 18 hours). The solvent was removed under reduced pressure. The residue was diluted with CH₂Cl₂ (30 mL) and washed with 3N HCl (20 mL×3). Next, the organic layer was dried over anhydrous Na₂SO₄, filtered, and evaporated to give the crude compound as an orange solid. The crude product was further purified by silica gel column to get the desired compound as a white solid (179 mg pure product, HPLC 97%, confirmed by H-NMR and MS; 500 mg of mixture, yield:50%).

### Preparation of Compound C0030

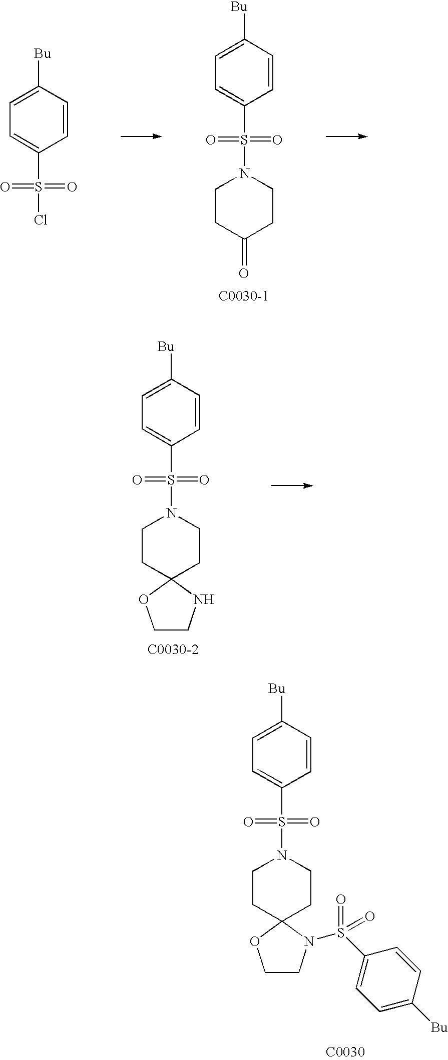

### Compound C0030-1

To a solution of piperdin-4-one (594 mg, 3.9 mmol) in 20 mL of pyridine was added 4-n-butylbenzenesulfonyl chloride (600 mg, 2.6 mmol). The mixture was stirred overnight (about 18 hours) at room temperature. The solvent was removed under reduced pressure. The residue was then diluted with 50 mL of dichloromethane, washed with 1N hydrochloride (30 mL×3). Next, the organic layer was dried and concentrated to give the crude product as a white solid (501 mg, yield: 66%, ¹HNMR confirmed).

### Compound C0030-2

A solution of C0030-1 (500 mg, 1.7 mmol), 2-aminoethanol (5 mL) and p-toluenesulfonic acid monohydrate (100 mg) in 30 mL of ethanol was stirred at 25° C. overnight (about 18 hours). The solvent was removed by reduced pressure evaporation. The residue was diluted with 50 mL dichloromethane, washed with water (50 mL×3) and saturated sodium bicarbonate aqueous (50 mL×3). The organic layer was dried and concentrated to give the product as a yellow solid (200 mg, yield: 89%, ¹H-NMR confirmed).

### Compound C0030

To a solution of C0030-2 (512 mg, 1.5 mmol) in 20 mL of pyridine, 4-n-butylbenzenesulfonyl chloride (528 mg, 2.3 mmol) was added with stirring at room temperature overnight (about 18 hours). The solvent was then removed under reduced pressure. The residue was diluted with 50 mL dichloromethane and washed with 1N hydrochloride (30 mL×3). Next, the organic layer was dried and concentrated to get the crude product as a brown oil (796 mg).

### Preparation of Compounds C0032 and C0033

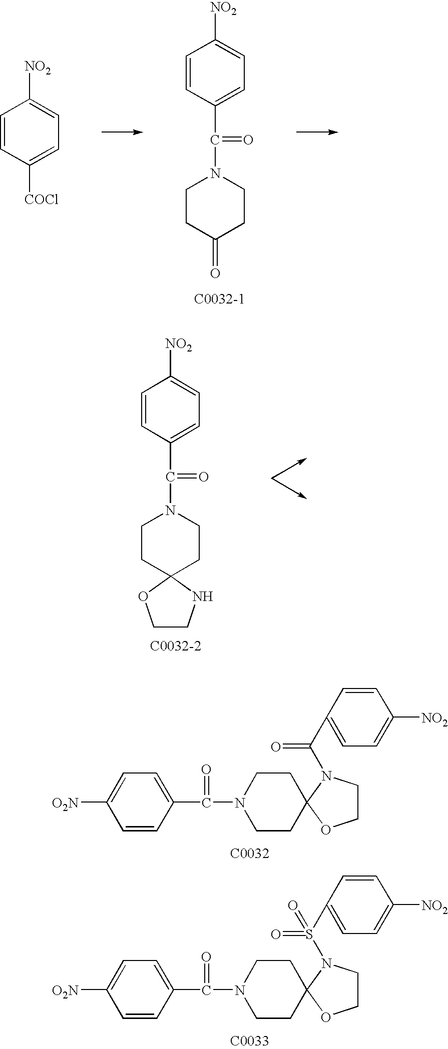

### Compound C0032-1

To a solution of piperidine-4-one (3.15 g, 10.17 mmol) in pyridine (30 mL), p-nitrobenzoyl chloride (2 g, 10.87 mmol) was added. The reaction mixture was stirred at room temperature overnight (about 18 hours). The solvent was removed under reduced pressure. The residue was diluted with CH₂Cl₂ (30 mL) and washed with 3N HCl (20 mL×3). The organic layer was dried over anhydrous Na₂SO₄, filtered, and evaporated to give the crude compound as a yellow solid (1.49 g, yield: 55.9%, confirmed by ¹H-NMR and LCMS).

### Compound C0032-2

A solution of compound C0032-1 (2 g, 8.06 mmol), 2-aminoethanol (0.73 g) and p-toluenesulfonic acid monohydrate (200 mg) in ethanol (40 mL) was stirred at 25° C. overnight (about 18 hours). The solvent was removed under reduced pressure. The residue was diluted with CH₂Cl₂ (30 mL) and washed with NaHCO₃ (30 mL×3). Next, the organic layer was dried over anhydrous Na₂SO₄, filtered, and evaporated to give the crude compound as an orange solid. (2.2 g, yield: 93.7%, confirmed by ¹H-NMR and LCMS).

### Compound C0032

To a solution of compound C0032-2 (1.07 g, 3.68 mmol) in pyridine (30 mL), 4-nitro-benzoyl chloride (1.02 g, 5.52 mmol) was added. The reaction mixture was stirred overnight (about 18 hours) at room temperature.

### Compound C0033

To a solution of compound C0032-2 (1.12 g, 3.85 mmol) in pyridine (30 mL), 4-nitrobenzene-sulfonyl chloride (1.28 g, 5.77 mmol) was added. The reaction mixture was stirred overnight (about 18 hours) at room temperature.

### Preparation of Compound C0034

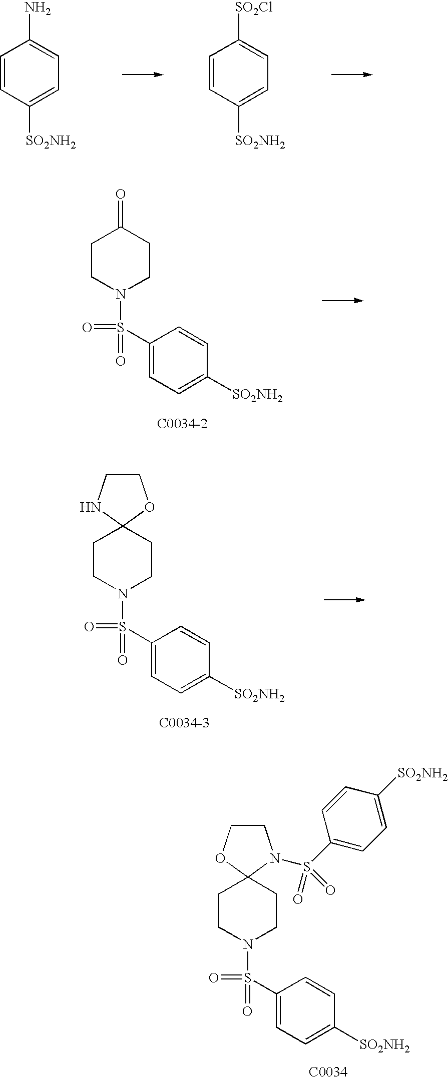

### Compound C0034-1

Cupric chloride (5 g) was added to a saturated solution of sulfur dioxide in CH₃COOH (200 mL) and sulfur dioxide gas (from the reaction of NaHSO₄ and H₂SO₄). The gas was slowly bubbled into the solution for 4 hours until the solution became blue-green colored. Next, 4-amino-benzene-1-sulfonamide (20 g, 116 mmol) was added to a solution of concentrated HCl (40 mL) and H₂O (50 mL) with stirring for 1 hour at 0° C. To this mixture was added a solution of sodium nitrate (8 g, 116 mmol) at such a rate of addition that the temperature did not rise above 0° C. The mixture was stirred for 0.5 hours then quenched with the SO₂/CuCl₂ solution made earlier. The mixture was then stirred for 1 hour at room temperature. Next, H₂O (500 mL) was added, and stirring continued for an additional 30 minutes. The product was collected by suction filtration, washed with H₂O, dried in vacuo at 60° C. to give the title product as a light yellow solid (LC-MS confirmed). After drying, about 10 g crude product as a light yellow solid was obtained (10 g, yield: 33%, confirmed by LC-MS).

### Compound C0034-2

To a solution of piperidine-4-one (0.72 g, 4.7 mmol) in 20 mL pyridine, was added compound C0034-1 (1.00 g, 3.9 mmol). The mixture was stirred overnight (about 18 hours) at room temperature. The solvent was then removed under evaporation. The residue was diluted with DCM (100 mL) and washed with 3M HCl (50 mL×3). The separated organic layer was dried over anhydrous Na₂SO₄ then evaporated to give the crude product as a white solid. (0.31 g, yield: 24.9%, TLC confirmed).

Repeat: A solution of piperidine-4-one (1.4 g, 9.4 mmol)in 30 ml pyridine was added compound C0034-1(2.00 g,7.8 mmol). The mixture was stirred overnight (about 18 hours) at room temperature. The solvent was removed under reduced pressure and the residue was diluted with CH₂Cl₂. The crude product was washed with 2N HCl (50 mL×3). The aqueous layer was extracted with CH₂Cl₂. The organic phase was combined and concentrated to give the crude product as a light yellow solid (0.65g, yield: 37%, TLC confirmed)

### Preparation of C0034-3

To a solution of compound C0034-2 (0.5 g, 1.58 mmol) in 10 mL ethanol was added ethanolamine (5 ml) and 4-methylbenzenesulfonic acid monohydrate (0.1 g). The mixture was stirred overnight (about 18 hours) at 25° C. Then the solvent was removed under reduced pressure. The residue was diluted with CH₂Cl₂ (100 mL), and washed with saturated NaHCO₃ (50 mL×6), there was much dissolved solid. Then the organic phase was dried over anhydrous Na₂SO₄ and concentrated to give few yellow solid. The aqueous layer was filtered to provide a white solid, checked it by NMR. The aqueous layer was extracted with CH₂Cl₂ until there was no fluorescence under UV in the new extraction. The white solid was confirmed to be the product, which was purified with silica gel column to give the pure product as white solid (0.25 g, yield: 43.9%, ¹H-NMR confirmed).

### Compound C0034

To a solution of compound C0034-3 (0.245 g, 0.68 mmol) in 20 ml pyridine was added C0034-1 (0.257 g, 1.01 mmol). The mixture was stirred at room temperature overnight (about 18 hours). The solvent was removed and the residue was diluted with CH₂Cl₂ (50 mL), washed with 2N HCl (50 mL×3). There was some solid dissolved in both aqueous phase and organic phase. The two phases were combined and filtered, to provide some yellow solid. The aqueous phase was extracted with CH₂Cl₂ (50 mL×3), and then concentrated to give some white solid. The NMR showed that the yellow solid contained compound C0034. The yellow solid was purified by chromatography on silica gel (CH₂Cl₂:CH₃OH=200:1) to give compound C0034 as a white solid (50 mg, yield: 12.7%, ¹1H-NMR and MS confirmed, HPLC 97%).

### Preparation of Compound C0041

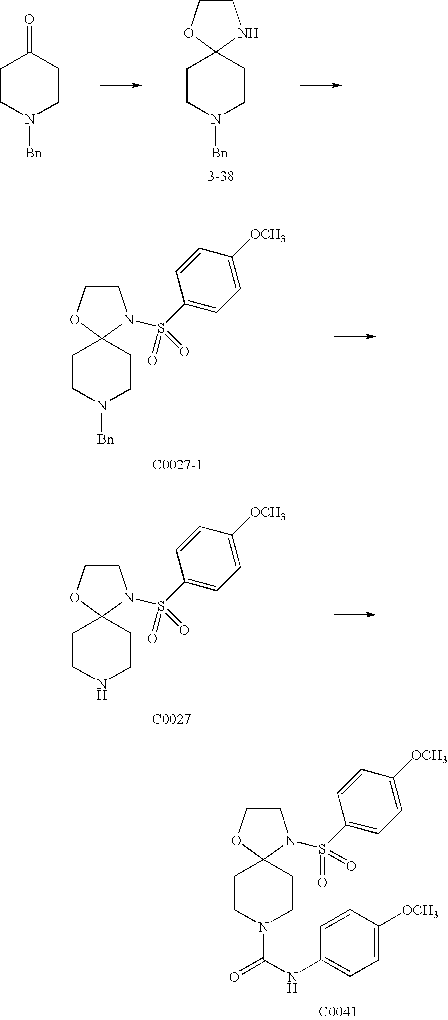

### Compound C0027

To the solution of C0027-1 (410 mg, 1.02 mmol) in MeOH:DCM=2:1 (30 mL), 10% Pd/C (0.2 g) was added, the reaction mixture was stirred at room temperature overnight under H₂. The solvent was filtered to remove Pd/C. The solvent was removed under the reduced pressure to give the white foam as product (310 mg, yield: 98%, confirmed by LCMS).

### Compound C0041

To the solution of C0027 (90 mg, 0.29 mmol) in CH₂Cl₂ (5 mL), 4-methoxyphenyl isocyanate (0.06 mL, 0.43 mmol) was added. The reaction mixture was stirred at room temperature overnight (about 18 hours). The reaction mixture was evaporated to removed the solvent, the residue was purified with silica gel column. TLC showed there were five spots, the spot of desired compound is weak (confirmed by LCMS).

### Preparation of C0042

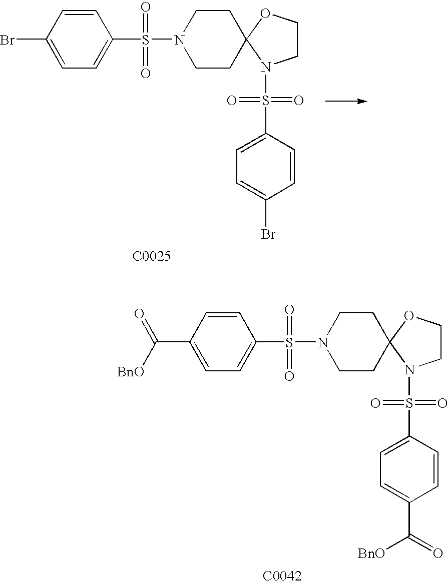

To a solution of compound C0025 (400 mg, 0.69 mmol) in 20 mL of DMF was added Pd(PPh₃)₄ (239 mg, 0.21 mmol), triethylamine (0.3 mL, 2.07 mmol) and 8 mL of benzyl alcohol. The mixture was stirred at 130° C. for 2 days under CO gas (P=2.5Mpa). The solvent was removed under reduced pressure, then the residue was diluted with methanol (25 mL) and filtered to get the product as a yellow solid. After purification with a silica gel column using dichloromethane solvent, the desired product was obtained as a yellow solid (399 mg, Yield:83.7%, confirmed by LC-MS, the purity of 99 is confirmed by HPLC).

### Preparation of Compound C0047

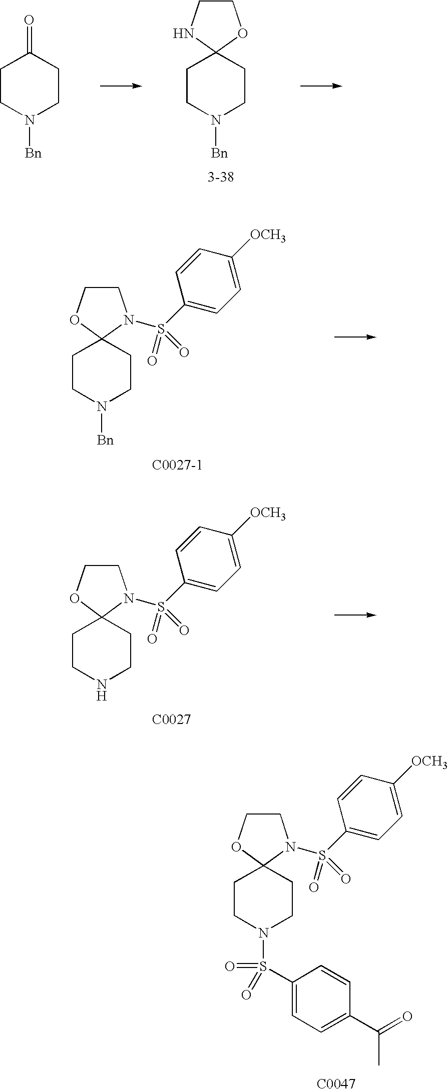

### Compound 3-38

To a solution of N-benzyl-piperidin-4-one (10 g 52.8 mmol) in 80 mL of ethanol, p-toluene-sulfonic acid monohydrated (100 mg), 2-aminoethanol (5 mL) was added; the mixture was stirred at 25° C. overnight (about 18 hours). The solvent was removed under the reduced pressure evaporation, the residue was diluted with 50 mL dichloromethane, and then washed with saturated sodium bicarbonate solutions (30 mL×3), saturated sodium carbonate (30 mL×3), then the organic layer was dried and concentrated to get the product as yellow oil (11.5 g, yield: 93.8).

### Compound C0027-1

To the solution of compound 3-38 (1.37 g, 5.91 mmol) in pyridine (20 mL) was added 4-methoxy-benzene-1-sulfonyl chloride (1.83 g, 8.85mmol). The reaction mixture was stirred overnight (about 18 hours) at room temperature. The solvent was removed under reduced pressure. The residue (brown oil) was purified with silica gel column to give yellow foam (410 mg, yield: 17%, confirmed by LC-MS).

### Compound C0027

To a solution of C0027-1 (0.334 g, 0.83 mmol) in 20 mL methanol was added 70 mg Pd(OH)₂ with stirring at 50° C. under H₂ (p=2.5 Mpa) for 2 days. The mixture was then filtered to remove the Pd(OH)₂/C and the filtrate was evaporated to give the crude product. The crude product was purified on a silica gel column (eluted with DCM:MeOH from 100:1 to 50:1) to give the purified compound as white solid (210 mg, yield: 81.1%, confirmed by LC-MS and ¹HNMR).

### Compound C0047

To the solution of C0027 (0.21 g, 0.67 mmol) in 20 mL pyridine was added 4-acetylbenzene-sulfonyl chloride (0.162 g, 0.74 mmol). The mixture was stirred at room temperature overnight (about 18 hours). The solvent was removed by reduced pressure evaporation, and the residue was diluted with 50 mL dichloromethane, then washed with 1M HCl three times (30 mL). The organic layer was dried over anhydrous Na₂SO₄ then concentrated to give the crude product as a yellow solid. After purification on a silica gel column (DCM:MeOH=500:1 to 250:1), the product was obtained as a white solid (0.224 g, yield: 67.5%, confirmed by LC-MS, ¹HNMR and MASS, HPLC 99%).

### Preparation of C0052

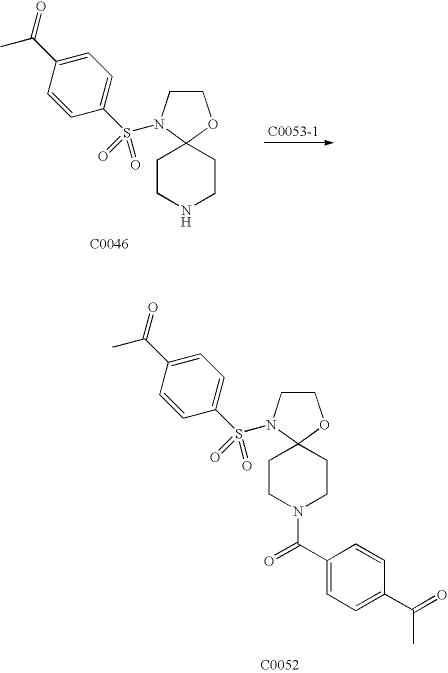

To a solution of C0046 (190 mg, 0.586 mmol) in dry DCM (20 mL) and DIEA (0.5 mL) was added dropwise a solution of 4-acetylbenzoyl chloride (128 mg, 0.703 mmol) in dry DCM (8 mL) at 0° C. After the addition, the mixture was stirred overnight (about 18 hours) at room temperature. The mixture was then washed with water (30 mL×3), the organic layer was dried and evaporated to give the crude product as a yellow solid. The crude product was purified with silica gel column to yield the pure product as white solid (135 mg, yield: 49%, confirmed by LCMS, NMR and MS, HPLC: 98.7%).

### Preparation of C0053 and C0054

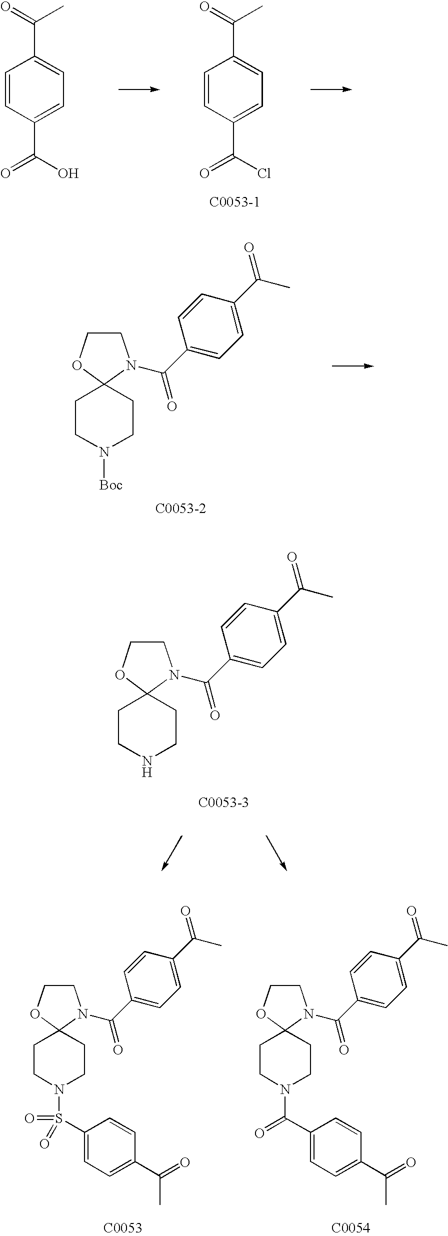

### Compound C0053-1

To a solution of 4-acetylbenzoic acid (250 mg, 1.52 mmol) in dry DCN (20 mL) and DMF (0.1 mL) was added dropwise oxalyl chloride (570 mg, 4.5 mmol) at 0° C. After addition the mixture was stirred for 2 hours at room temperature. The solvent and excess oxalyl chloride was removed by reduced pressure evaporation to give the product as a yellow solid (270 mg, yield: 97%, confirmed by LCMS dissolved with MeOH.

### Compound C0053-2

To a solution of C0011-1 (727 mg, 23 mmol) and DIEA (1 mL) in dry DCM (20 ml) was added C0053-1 (500 mg, 2.74 mmol solution in 20 mL dry DCM) dropwise at 0° C. Next, the mixture was stirred at room temperature for 3 days. The mixture was then washed three times with water (50 mL), the organic layer was dried then evaporated to get the product as brown oil (1.28 g, yield:100%, confirmed by LCMS).

### Compound C0053-3

A solution of C0053-2 (1 g, 2.58 mmol) and CF₃COOH (5 mL) in DCM (20 mL) was stirred overnight (about 18 hours) at room temperature. The mixture was washed with saturated Na₂CO₃ solution, the organic layer was dried and evaporated to give the crude product as a brown oil. The crude product was purified on a silica gel column to provide the purified product as a brown oil (360 mg, yield: 48.3%, confirmed by LCMS.)

### Compound C0053

To a solution of C0053-3 (160 mg, 0.55 mmol) in pyridine (20 mL) was added 4-acetylbenzene-1-sulfonyl chloride (120 mg, 0.55 mmol). The mixture was stirred overnight (about 18 hours) at room temperature. The solvent was removed by reduced pressure evaporation. The crude product was diluted with 50 mL DCM and washed three times with 1N HCl (30 mL). The organic layer was dried and evaporated to give the crude product as a yellow solid. Purification with silica gel column gave the pure product as a white solid (102 mg, yield: 39%, confirmed by LCMS, MS and NMR: HPLC: 95.26%).

### Compound C0054

To a solution of C0053-3 (160 mg, 0.55 mmol) and DIEA (0.3 ml) in dry DCM (20 mL) was added dropwise a solution of 4-acetylbenzoyl chloride (111 mg, 0.61 mmol) in dry dichloromethane (8 mL) at 0° C. After the addition, the mixture was stirred at room temperature for 2 days. To this mixture was added 20 ml of DCM, then the mixture was washed with water (40 ml×3). The organic layer was dried and evaporated to give the crude product as a yellow oil. Purification with silica gel column gave the pure product as white solid (110 mg, yield: 45.7%, confirmed by LCMS, MS and NMR. HPLC: 99.74%).

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A compound of Formula I 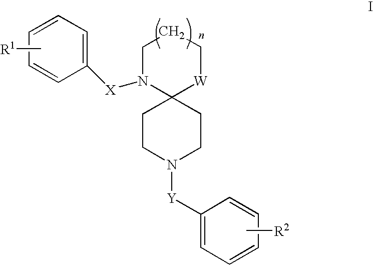
wherein
X and Y are the same or different and are SO₂, C(O) or NHC(O);
W is NR⁷ or O, where R⁷ is H, C₁-C₆ hydrocarbyl, or C₁-C₇ acyl;
n is zero or one; and
R¹ and R² are the same or different and are selected from the group consisting of H, C₁-C₆ hydrocarbyl, C₁-C₆ hydrocarbloxy, trifluoromethyl, trifluoromethoxy, C₁-C₇ acyl, C₁-C₆ hydrocarbylsulfonyl, halogen, nitro, phenyl, cyano, carboxyl, C₁-C₇ hydrocarbyl carboxylate, carboxamide wherein the amido nitrogen has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, and NR⁵R⁶ wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur;
with the proviso that R¹ and R² are not both methoxy when X and Y are both SO₂, W is O and n is zero.

2. The compound according to claim 1, wherein X and Y are the same.

3. The compound according to claim 2, wherein X and Y are both SO₂.

4. The compound according to claim 1, wherein W is O.

5. The compound according to claim 1, wherein R¹ and R² are the same.

6. The compound according to claim 5, wherein R¹ and R² have a Hammett sigma value for the para-position greater than −0.2.

7. The compound according to claim 5, wherein R¹ and R² are present at the same relative position in each of their respective rings relative to the position of the X and Y groups, respectively.

8. The compound according to claim 7, wherein R¹ and R² are selected from the group consisting of trifluoromethyl, C₁-C₆ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

9. The compound according to claim 1, wherein n is zero.

10. A compound of Formula II 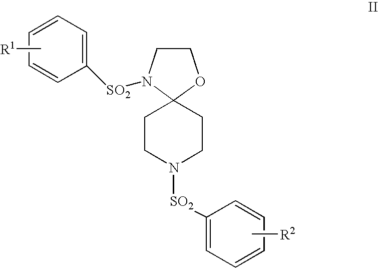
wherein
R¹ and R² are the same and are selected from the group consisting of trifluoromethyl, C₁-C₆ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

11. The compound according to claim 10 wherein said compound of Formula II is selected from the group consisting of

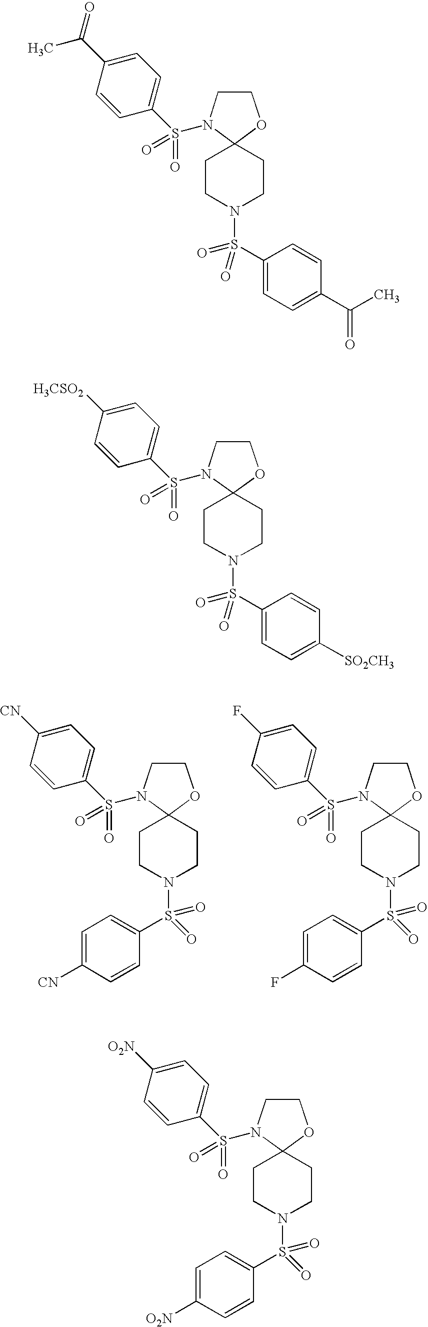

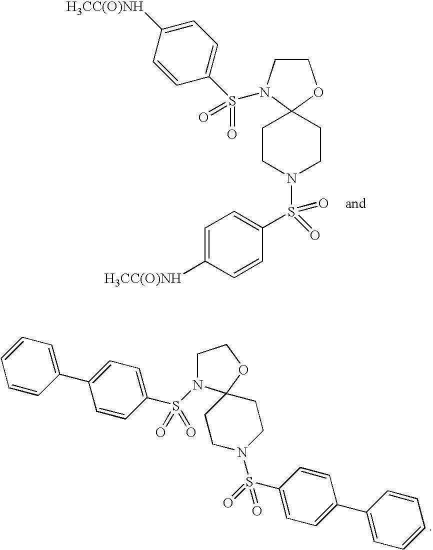

12. A compound of Formula III 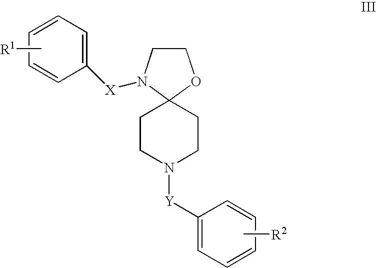
wherein
X and Y are both C(O), X is SO₂ and Y is C(O), or X is SO₂, and Y is NHC(O); and
R¹ and R² are the same and are selected from the group consisting of trifluoromethyl, C₁-C₆ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

13. The compound according to claim 12 wherein said compound of Formula III is

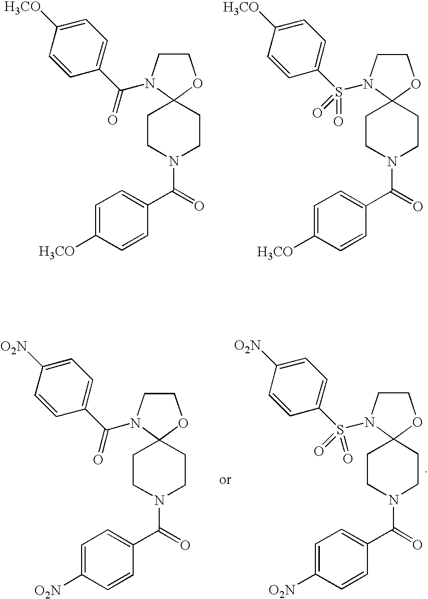

14. A pharmaceutical composition comprising an analgesic effective amount of a compound of claim 1 dissolved or dispersed in a physiologically tolerable carrier.

15. The pharmaceutical composition according to claim 14 wherein said compound is a compound of claim 10.

16. The pharmaceutical composition according to claim 14 wherein said compound is a compound of claim 12.

17. A method of reducing pain in a host mammal in need thereof that comprises administering to that host mammal a pharmaceutical composition containing an analgesic effective amount of a compound of Formula IV dissolved or dispersed in a physiologically tolerable carrier 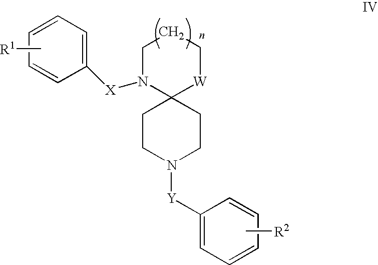
wherein
X and Y are the same or different and are SO₂, C(O) or NHC(O);
W is NR⁷ or O, where R⁷ is H, C₁-C₆ hydrocarbyl, or C₁-C₇ acyl; and
R¹ and R² are the same or different and are selected from the group consisting of H, C₁-C₆ hydrocarbyl, C₁-C₆ hydrocarbyloxy, trifluoromethyl, trifluoromethoxy, C₁-C₇ acyl, C₁-C₆ hydrocarbylsulfonyl, halogen, nitro, phenyl, cyano, carboxyl, C₁-C₆ hydrocarbyl carboxylate, carboxamide wherein the amido nitrogen has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, and NR⁵R⁶ wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

18. The method according to claim 17, wherein X and Y are the same.

19. The method according to claim 18, wherein X and Y are both SO₂.

20. The method according to claim 17, wherein W is O.

21. The method according to claim 17, wherein R¹ and R² are the same.

22. The method according to claim 21, wherein R¹ and R² have a Hammett sigma value greater than zero.

23. The method according to claim 21, wherein R¹ and R² are present at the same relative position in each of their respective rings relative to the position of the X and Y groups, respectively.

24. The method according to claim 17, wherein said host mammal is selected from the group consisting of a primate, a laboratory rodent, a companion animal, and a food animal.

25. The method according to claim 17, wherein said compound is a compound of Formula II 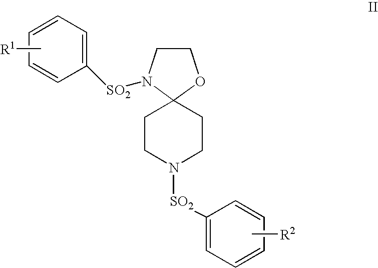
wherein
R¹ and R² are the same and are selected from the group consisting of C₁-C₆ alkoxy, trifluoromethyl, C₁-C₇ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

26. The method according to claim 17, wherein said composition is administered a plurality of times over a period of days.

27. The method according to claim 26, wherein said composition is administered a plurality of times in one day.

28. The method according to claim 17, wherein said composition is administered perorally.

29. The method according to claim 17, wherein said composition is administered parenterally.

30. The method according to claim 17, wherein said compound is a compound of Formula III 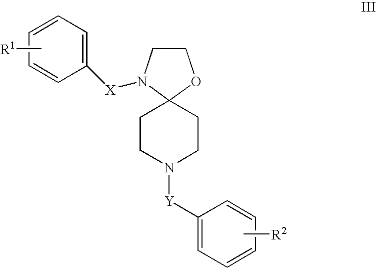
wherein
X and Y are both CO or X is SO₂ and Y is CO; and
R¹ and R² are the same and are selected from the group consisting of C₁-C₆ alkoxy, trifluoromethyl, C₁-C₇ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.
